(19) United States
(12) Patent Application Publication
(10) Pub. No.: US 2024/0181089 A1
(43) Pub. Date: Jun. 6, 2024

(54) PHARMACEUTICAL POLYMER CONJUGATES

(71) Applicant: XYLONIX PTE. LTD, Singapore (SG)

(72) Inventor: Jinhyuk Fred Chung, Singapore (SG)

(21) Appl. No.: 18/282,094

(22) PCT Filed: Mar. 16, 2022
(86) PCT No.: PCT/SG2022/050139

§ 371 Date: Sep. 14, 2023

(30) Foreign Application Priority Data
Mar. 18, 2021 (SG) 10202102785S

(51) Int. Cl. A61K 49/00; A61K 47/64; A61P 35/00

(52) U.S. Cl. CPC A61K 49/0056; A61K 47/641; A61K 49/0032; A61P 35/00

(57) ABSTRACT

The present invention relates to compositions for the intracellular delivery of therapeutically, active divalent metal ions, the compositions comprising a polypeptide having (i) one or more targeting moiety conjugated thereto, (ii) an ionophore conjugated thereto via a cleavable linker, and (iii) a divalent metal ion bound thereto, and methods for preparing such compositions, and their use in therapeutic methods.

### TECHNICAL FIELD

Presented herein are polypeptide polymer conjugates useful for the intracellular delivery of a therapeutically effective amount of a metal ion. The invention also relates to compositions for inducing parthanatos in cells to which the composition is directed, and methods of treatment using such compositions.

### BACKGROUND

The complexity and heterogeneity of cancer has long been recognized, yet for some time the treatment modalities followed the historical development of, for example, disease classifications according to the physiological location, histological appearance, and lineage. Recently, attention has turned to immunotherapies and other immune-related approaches, such as initiating an anti-cancer T cell immune response through vaccination or induction of immunogenic cell death as a means for causing neo-antigen presentation in tumor cells.

Several mechanisms may be relevant to methods for inducing immunogenic cell death, such as necroptosis, pyroptosis, or parthanatos. Whereas there are several pre-clinical studies demonstrating the potential for necroptosis-based therapies, little progress has been reported for methods that exploit pyroptosis or parthanatos pathways. Parthanatos is a mode of programmed necrosis triggered by hyperactivation of the DNA damage sensor and repair enzyme, PARP. The excessive activation of PARP causes the reaction product, PAR polymer, to accumulate, which leads to nuclear AIF translocation, which in turn triggers severe DNA fragmentation and, ultimately, cell death.

Reports have suggested that acute zinc or cadmium toxicity involve PARP1 activity. (NPL1, 2). For example, a report assessing neurotoxicity of zinc salts describes that high concentrations of zinc ion from simple zinc salts (400 μM or 26 μg/mL) induces PARP/PARG-mediated NAD+ and ATP depletion and subsequent necrosis in cultured cortical cells. (NPL3). Conversely, a study of zinc activity against a cancer cell line observed certain necrotic mechanisms, but at concentrations of the zinc agent that had been shown by others to cause acute neurological toxicity in rats. (NPL4, 5).

Other studies with these divalent metal ions have focused on their pyrithione (“Pyr”) complexes (i.e., ZnPyr₂). Although one study reported that ZnPyr₂ exhibits cytotoxic effects and thus potentially has antitumor properties, that study found ZnPyr₂ induced apoptosis in cancer cell lines, and caused complete PARP cleavage while not causing any DNA damage. (NPL6). Apoptotic cell death is considered to be orthogonal (lack any mechanistic commonality) to the parthanatos mechanism, indeed, the PARP cleavage reported for ZnPyr₂ would prevent any PAR activity, let alone hyperactivity, which serves to trigger the parthanatos cascade.

We have previously disclosed the anticancer efficacy of digestible polymer-zinc chelate complexes (zinc γ-polyglutamate [Zn-γPGA] and zinc α-polyglutamate [Zn-αPGA], respectively) in Singapore patent application numbers 10201609131Y, filed 1 Nov. 2016, and 10201708886R, filed Oct. 30, 2017. And subsequently, in Singapore patent application numbers 10201805412T, filed 22 Jun. 2018, and 10201811577T, filed 24Dec. 2018, we disclosed that related zinc-based agents have a therapeutic benefit, ether as monotherapy or in combination with immune-oncology agents against a range of cancer indications. The foregoing applications are hereby incorporated by reference in their entirety.

Nonetheless, there remains a need for improved agents that either demonstrate greater efficacy against tumors and/or activity against other cell types, such as M2-like macrophages.

To solve the problems referred to we experimented with the design of polypeptide polymers, and how additional structural features and functional properties might improve the ability of metal ions to exert therapeutic effects within a cell, particularly the cell types targeted by the polypeptide conjugates used to deliver the metal ions. We have experimented with the types of moieties conjugated thereto, and their manner of conjugation to prepare polymer conjugate compositions useful for the intracellular delivery of divalent metal ions, that are, furthermore, capable of inducing parthanatos. We tested such compositions for activity against cancer cells (including solid tumor cancer and blood cancer cells) and M2-like macrophages, and found such compositions to have a potent tumoricidal effect and to induce parthanatos, and, accordingly, completed our invention as described herein.

Compositions and methods disclosed herein result from the surprising observation that metal ion complexes of polypeptide polymer conjugates comprising a targeting moiety and a cleavable ionophore moiety can induce parthanatos in various human and mouse tumors and can initiate a response in antitumor immune compartments such as T cells and macrophages in in vivo testing.

### CITATIONS

NPL1. Sheline, C. T., Wang, H., Cai, A. L., Dawson, V. L., Choi, D. W. (2002). Involvement of poly ADP ribosyl polymerase-1 in acute but not chronic zinc toxicity. Eur. J. Neurosci. 18, 1402-1409.
NPL2. Luo, T., Yuan, Y., Yu, Q., Liu, G., Long, M., Zhang, K., Bian, J., Gu, J., Zou, H., Wang, Y., Zhu, J., Liu, X., Liu, Z. (2017). PARP-1 overexpression contributes to cadmium-induced death in rat proximal tubular cells via parthanatos and the MAPK signalling pathway. Scientific Reports 7, 4331.
NPL3. Kim, Y. H., and Koh, J. Y. (2002). The role of NADPH oxidase and neuronal nitric oxide synthase in zinc-induced poly(ADP-ribose) polymerase activation and cell death in cortical culture. Experimental Neurology 177, 407-418.
NPL4. Carraway, R. E., and Dobner, P. R. (2012). Zinc pyrithione induces ERK- and PKC-dependent necrosis distinct from TPEN-induced apoptosis in prostate cancer cells. Biochimica et Biophysica Acta 1823, 544-557.
NPL5. Snyder, D. R., de Jesus, C. P., Towfighi, J., Jacoby, R. O., and Wedig, J. H. (1979). Neurological, microscopic and enzyme-histochemical assessment of zinc pyrithione toxicity. Food and Cosmetics Toxicology 17, 651-660.
NPL6. Zhao, C. et al. (2017). Repurposing an antidandruff agent to treating cancer: zinc pyrithione inhibits tumor growth via targeting proteasome-associated deubiquitinases. Oncotarget 8, 13942-13956.

### SUMMARY

The present disclosure generally relates to therapeutically active polypeptide polymer conjugate compositions and methods of making and using them. More specifically, the compositions comprise a targeting moiety and an ionophore ligand conjugated to a polymer, and a therapeutically-active metal ion bound to said polymer, and such compositions are useful for the intracellular delivery to cells targeted by the composition of a therapeutically effective amount of the metal ion. The therapeutically active metal ions are selected from zinc(II) and cadmium(II) ions.

In one aspect the present disclosure provides polypeptide polymer conjugate compositions and their methods of synthesis. The polymer is comprised of monomer units joined via peptide bonds, and the monomer units include a side chain with a functional group available for conjugating various functional moieties to the polymer. In one preferred embodiment, the functional group present in the monomer unit side chain is a carboxyl group. A carboxyl group provides a wide variety of well-known conjugation chemistries for joining various moieties to the polymer backbone. Also, this functional group can be prepared in its carboxylate form, and used to bind therapeutically-active metal ions to the polymer.

At least one monomer unit side chain is conjugated to a targeting moiety and at least one monomer unit side chain is conjugated to an ionophore moiety. The targeting moiety comprises a molecule that is capable of being recognized by cell-surface receptors found on cells to which the composition is directed. The moiety thus serves to guide the polymer composition to the particular cells whereupon the receptor may facilitate uptake of the polymer composition into the cell. The ionophore comprises a molecule that is capable of forming a coordination complex with the therapeutically-active metal ion. Generally, conjugation of a functional moiety is accomplished by joining the side chain functional group with the functional moiety via a linker group. Preferably, a targeting moiety is joined to the polymer via a non-cleavable covalently bonded linker group, whereas an ionophore moiety is joined to the polymer via a covalently bonded linker group that contains a cleavable bond, which thereby permits the ionophore moiety to separate from the polymer backbone and form a complex with the therapeutically-active metal ion.

Among the remaining monomer units that are not conjugated to either a targeting moiety or an ionophore moiety, in some embodiments the side chains in such monomer units are independently in protonated (carboxylic acid) or non-protonated (carboxylate ion) form. The form of the carboxyl group generally depends on the chemical workup that produces a solid form of the polymer—the polymer may prepared as a salt or as the free acid, or whether the polymer is provided in a solution, in which case the carboxyl groups' ionization state in aqueous solutions is a function of the pH. In other embodiments, a plurality of the remaining non-conjugated side chains are present as the carboxylate ion and are complexed to a therapeutically-active metal ion, while the other remaining side chain carboxyl groups may be present in protonated and/or non-protonated forms.

In some embodiments, the polymer conjugates of the invention comprise a partial structure illustrated by formula (I):

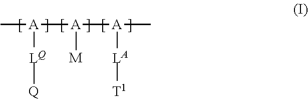

where:
A is a monomer unit bearing a side chain having an ionizable functional group;
L^{Q} is a cleavable linking group;
Q is an ionophore ligand
L^{A} is a first linking group;
T¹ is a first targeting moiety that targets a first receptor;
M is independently selected at each occurrence and may be a proton, a cationic counterion, or a therapeutically-active metal ion; and
the brackets represent one or more occurrences of each type of monomer unit that collectively form the polymer. The number of each monomer type is independent of one another, but the number of occurrences for a particular monomer type may be selected according to the functionality and properties desired of the composition, as described herein. No primary structure is intended by the illustration, as the occurrence of each monomer type is generally randomly ordered, as those of skill in the art understand, particularly in view of the disclosure herein.

In other embodiments, the polymer conjugates of the invention comprise a partial structure illustrated by formula (II):

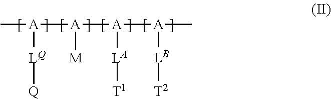

where:
the symbols in common with formula (I) have the same meaning, and
L^{B} is a second linking group;
T² is a second targeting moiety that targets the first receptor or a second receptor.

In other embodiments, the polymer conjugates of the invention comprise a partial structure illustrated by formula (III):

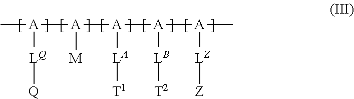

where:
the symbols in common with formulas (I) and (II) have the same meaning, and
L^{Z} is a label linking group;
Z is a label moiety.

The label moiety is a detectable label that serves to facilitate chemical synthesis development or in vitro, in situ, or in vivo investigative studies. Preferably, the label is a fluorophore.

In each of formulas (I)-(III) it should recognized that to the extent that the polymer backbone has a terminal functional group that is the same as or has similar chemical reactivity as the side chain functional group, one the linking groups L^{A}, L^{B}, L^{Q}, or L^{Z}, may bond to the terminal functional group position. This may be the result of random competition, the order of addition of reagents in a conjugation reaction, or a reactivity preference due to the coupling agent, the reaction conditions, or the functional group of the particular linker group.

In another aspect, the invention provides any of the above polymer compositions of formulas (I)-(III) prepared with a therapeutically-active metal ion present, as a therapeutically-active polymer conjugate agent. Such therapeutic agent compositions are generally formulated as liquid solutions, and in a pharmaceutically acceptable manner consistent with the route and form of administration. In one embodiment, the therapeutic agent compositions are formulated for intravenous administration.

In another aspect, provided herein is the use of pharmaceutical compositions of any of the embodiments of a therapeutically-active polypeptide polymer conjugate in methods for treating solid tumors or blood cancers in a subject. In some embodiments, the solid tumors or blood cancers treated are those types that are susceptible to PARP-mediated necrotic death.

In another aspect, provided herein is the use of pharmaceutical compositions of any of the embodiments of a therapeutically-active polypeptide polymer conjugate in methods for treating macrophage-mediated inflammations. In some embodiments, the pharmaceutical compositions are useful for targeting M2-like macrophages and treating macrophage-mediated inflammatory conditions. In some embodiments, the pharmaceutical compositions are useful for initiating an immune response in various immune compartments.

In another aspect, provided herein is the use of pharmaceutical compositions of any of the embodiments of a therapeutically-active polypeptide polymer conjugate in methods for treating conditions in a subject in which cells causing a pathology are susceptible to targeting by a folate moiety because the cells overexpress folate receptors or to targeting by a ligand of an integrin because the cells overexpress the targeted integrins.

In another aspect, provided herein is the use of a polypeptide polymer conjugate composition according to any of the embodiments disclosed herein in the manufacture of a pharmaceutical composition or medicament for use in the methods of treatment disclosed herein.

### BRIEF DESCRIPTION OF THE FIGURES

FIG. 1 shows one embodiment of a polymer conjugate that is described in Example 6.

FIG. 2 shows one embodiment of a polymer conjugate that is described in Example 7.

FIG. 3 shows one embodiment of a polymer conjugate that is described in Example 8.

FIG. 4 shows a polymer conjugate used as a control that is described in Example 9.

FIG. 5A shows comparative in vitro cytotoxicity evaluation using LDH release assays after 24 h treatment against 4T1 cells.

FIG. 5B shows results of the in vitro time-resolved apoptosis-necrosis flow cytometry assay.

FIG. 5C shows results of the in vitro dose-resolved apoptosis-necrosis flow cytometry assay.

FIG. 5D shows in vitro PAR-ELISA assay results on the CO1ODS-Zn treated 4T1 cells with or without the PARP inhibitor PJ34.

FIG. 6A shows representative confocal fluorescence images of 4T1 cells treated for 1.5 h.

FIG. 6B shows quantitative analysis of the fluorescence images for cell viability, nuclear AIF translocation, and nuclear TUNEL intensity.

FIG. 7A shows ex vivo IC50 values of C010DS-Zn versus the 53 PDX-tumor types stratified by the tumor sites.

FIG. 7B shows plots of the ex vivo IC50 values versus the TMB or the MSI scores associated with each PDX-tumor fragment.

FIG. 7C shows an example ex vivo cytotoxicity response data from the CTG-1413 sarcoma PDX-tumor fragment.

FIG. 8 shows comparative fluorescence imaging characterization of anti-γ-H2AX uptake in the PDX tumor fragments upon ex vivo treatment with 10% DMSO (PC) or C010DS-Zn.

FIG. 9A shows 4T1-Balb/c treatment model scheme, tumor growth kinetics, and notable immune responses in the TME.

FIG. 9B shows CT26-Balb/c treatment model scheme, tumor growth kinetics, and notable immune responses in the TME.

FIG. 10A shows plasma pharmacokinetic profile of C010DS-Zn that separately traced C010DS via Cy5.5 signal and zinc levels after a single intravenous bolus injection of C010DS-Zn using Sprague-Dawley Rats.

FIG. 10B shows 13 days non-anticancer dosing scheme against 4T1-Balb/c model, its tumor growth kinetics, and the macrophage immune response to the treatment in the collected tumors.

FIG. 100 shows 16 days non-anticancer dosing scheme against 4T1-Balb/c model, its tumor growth kinetics, and the macrophage immune response to the treatment in the collected tumors.

### DETAILED DESCRIPTION

Polymer conjugates disclosed herein are comprised of two functional moiety types conjugated to the polymer backbone and are capable of forming complexes with therapeutically active metal ions. These components—a targeting moiety, an ionophore moiety, and a metal ion—when brought together as a metal ion polymer conjugate complex as described herein are capable of inducing a biological response in a subject that imparts a therapeutic benefit. The polymer conjugates serve to deliver a dose of a metal ion, with, it is believed, the assistance of the ionophore moiety, to the intracellular environment of cells that express a receptor to which the targeting moiety directs the polymer conjugate.

There are several general characteristics of the subject macromolecular compositions that contribute to its desirable properties. First, the compositions include a ligand that binds to a cell surface receptor. This ligand is referred to as a targeting moiety. Ligands of interest are those that bind to cell surface receptors that are overexpressed or at least abundant on the cell types of interest. Notably, tumor cells and immune cells, such as macrophages, are found to overexpress certain receptors, e.g., a folate receptor or folate binding protein, and thus offer a means to target these cell types using the receptor ligand, or analogs or derivatives thereof. Generally, the targeting moiety is covalently linked to the rest of the structure in a manner that is not susceptible to being cleaved. The link to the rest of the structure is generally hydrophilic, and sufficiently long to permit the ligand to approach the cell surface receptor with little steric interference from the rest of the structure. A plurality of targeting moieties may be linked to the structure, and/or more than one type of targeting moiety may be included.

Second, the compositions include an ionophore of the metal ion. This ionophore is referred to as an ionophore moiety. Ionophores are molecules that reversibly bind with a metal ion and aid the transport of the metal ion across a biological membrane. In a preferred embodiment the ionophore moiety is linked to the rest of the structure via a cleavable bond. In a further embodiment, the bond is cleaved as a result of the microenvironment in which the macromolecule accumulates. In a further embodiment, the ionophore is able to normally bind with the metal ion as a result of having been cleaved from the rest of the structure. That is, for example, the cleaving of the link exposes a functional group in the ionophore that is one of the groups that coordinates to the metal ion. A plurality of ionophore moieties may be linked to the structure, and/or more than one type of ionophore moiety may be included.

Third, the compositions include a metal ion that can cause a biological effect. The purpose of the compositions is to deliver metal ions within a subject so as to cause a biological effect having a therapeutic benefit. Generally, a plurality of metal ions are associated with each macromolecular structure, thus providing, in essence, a bolus dose of the metal ion to a particular cellular environment. The benefit of delivering the metal ions using the structures disclosed herein is that, without such structures, the metal ion would not otherwise be deliverable to the cellular environment at such concentration, and/or without such lack of toxic effect to the cell, tissue, organ, or subject as a whole.

Fourth, the compositions are comprised of a macromolecule that provides both (i) a scaffold upon which the various other components discussed above can bind to, so as to be deliverable as and function as a set, and (ii) sufficient size or bulk such that cells will uptake the composition by endocytosis (e.g., receptor-mediated endocytosis, adsorptive endocytosis, etc.). Common macromolecular structures that may provide such a scaffold include linear polymers, branched polymers, dendrimers, and other types of nanoparticles. Generally, such macromolecules should be water soluble, non-toxic, and non-immunogenic, in addition to providing the necessary functional groups to prepare conjugates and bind metal ions. Further, such macromolecules are preferably biodegradable, but if not, should be less than ˜40 kDa for efficient renal elimination. In the endocytotic uptake process, without being bound by theory, it is believed that endosomes and lysosomes will host the composition and that the microenvironments therein, such as, lower pH and the presence of digestive enzymes and redox active molecules (e.g., glutathione) can cause the cleavable link to the ionophore to cleave. As a result, the metal ion can bind with the ionophore, and the ionophore can assist the transport of metal ions from the endosomes and lysosomes into the rest of the intracellular environment, where the metal ion may exert the intended biological effect, such as triggering parthanatos.

These general principles and properties of the subject compositions having been described, reference will now be made in detail to certain embodiments of the invention.

### A. Polymer Conjugates

In one embodiment, the polymer conjugate is described by Formula (IV):

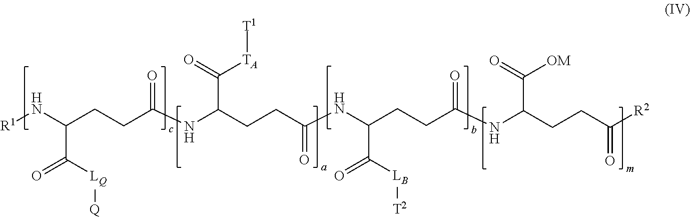

and metal ion complexes thereof.

In formula (IV), the polymer backbone is gamma-polyglutamic acid (γ-PGA), wherein the linear backbone is formed by peptide bonds between the amino group of one monomer and the carboxylic acid group located at the gamma position of the second monomer unit. As such, the carboxylic acid group at the alpha position of each monomer unit is a pendant side chain that is available to conjugate to, and is also capable of binding metal ions or pairing with cations when present as the carboxylate ion.

The formula features square brackets around the different monomer units, to indicate there are four different types of monomer units, potentially having distinct structures as defined by the substituents. Each bracket has a subscript (c, a, b, and m, from left to right), indicating that each monomer unit type may be present in that many instances. The formula does not, however, intend to require that the connectivity, that is, the primary structure, of the polymer backbone is “c” units of the first monomer type followed by “a” units of the second monomer type, and so on. Instead, the primary structure will comprise a random ordering of the various monomer types, subject to any tendencies that arise due to the synthetic route used to prepare the polymer conjugate composition, including, for example, the order of addition of the linking groups and side chains, the nature of the bonds formed between a linking group and the α-carboxyl group, coupling agents, reaction conditions, and the like, as understood by those skilled in the art.

Considering the terminal positions, at the N-terminal, R¹ is H, and at the C-terminal, R² is OH or OM or L_{A}-T¹ or L_{B}-T² or L_{Q}-Q.

T¹ and T² are targeting moieties. In one embodiment, T¹ and T² each bind with a different class of cell surface receptors, and thus represent different classes of ligands. In another embodiment, T¹ and T² are different molecules but they each bind with the same class of cell surface receptors. T¹ and T² may be a natural ligand (a molecule found in cells that is a natural binding partner with the receptor), or a ligand analog (a molecule not naturally found in cells that nonetheless has binding affinity for the receptor), or a ligand derivative (a modified form of the natural ligand, usually modified to facilitate conjugation). If T¹ or T² has a functional group present amenable to reacting to form a covalent bond, that functional group can be used to join the moiety to a linking group, or to directly conjugate the moiety to the polymer. Generally, a ligand analog will have been prepared to have a suitable functional group. A natural ligand, such as folate, may have a suitable functional group, but if not, a ligand derivative may be prepared so as to provide one.

T¹ and T² are joined to the polymer backbone through the α-carboxyl group in a pendant side chain, via a linking group L_{A} and L_{B}, respectively. L_{A} and L_{B} are any chemical moiety capable of linking the target moiety to the polymer backbone via covalent bonds. In the event that T¹ or T² can directly form a covalent bond with the polymer, then L_{A} or L_{B}, respectively, represent a bond. Otherwise, L_{A} and L_{B} represent a bifunctional molecule having a first terminal site capable of forming a bond with a pendant α-carboxyl group in a monomer unit, and a second terminal site capable of forming a bond with the suitable functional group in T¹ and T², respectively, wherein the first terminal site and the second terminal site are connected to one another through a chain of 3 to 20 atoms. The first terminal site may be an —O—, —S—, or an —NH—, thereby forming an ester, thioester, or amide link to the polymer. The second terminal site may be an —O—, —S—, —NH—, or an -acyl. The chain of 3 to 20 atoms may comprise a polyether, ether segments such as —O(CH₂CH₂)O— or an optionally substituted linear or branched hydrocarbon. The first terminal sites and the second terminal sites of L_{A} and L_{B} may be the same or different. For convenience, providing L_{A} and L_{B} with the same first terminal site may facilitate performing a coupling reaction between L_{A} and L_{B} and the polymer simultaneously.

At least one, or both, of T¹ and T² are present as a targeting moiety. The indices “a” and “b” are the degree of incorporation of T¹ and the degree of incorporation of T², respectively, wherein “a” and “b” represent the average number of such monomer units in the composition per polymer. Each of “a” and “b” may be zero or a finite number up to about 5, but “a” and “b” are not both zero.

Q is an ionophore moiety, comprising an ionophore to the metal ion used to prepare a metal ion complex of the polymer conjugate. In preferred embodiments, the ionophore is a bidentate ligand, and one of the ligands is a thiol or thione group, while the other ligand is generally an O- or N-based functional group. In one embodiment, the ionophore moiety is prepared so as to be joined to the linker L_{Q} via the thiol or thione group, and the linker L_{Q} is provided with a S-based functional group, whereby L_{Q}-Q are joined by a disulfide bond, which may be cleaved.

L_{Q} represents a linking group through which Q is joined to the α-carboxyl group of a monomer unit, and L_{Q} contains a cleavable site. The cleavable bond is that between L_{Q} and Q. L_{Q} represents a bifunctional molecule having a first terminal site capable of forming a bond with a pendant α-carboxyl group in a monomer unit, and a second terminal site capable of forming a bond with the suitable functional group in Q, wherein the first terminal site and the second terminal site are connected to one another through a chain of 3 to 20 atoms. The first terminal site may be an —O—, —S—, or an —NH—, thereby forming an ester, thioester, or amide link to the polymer. The cleavable terminal site may be an —S—, to, for example, form a disulfide link with a thiol group in Q. The chain of 3 to 20 atoms may comprise a polyether, ether segments such as —O(CH₂CH₂)O— or an optionally substituted linear or branched hydrocarbon. The first terminal site of L_{Q} may be the same or different as the first terminal sites of L_{A} and L_{B}. For convenience, providing L_{Q}, L_{A}, and L_{B} with the same first terminal site may facilitate performing a coupling reaction between L_{Q}, L_{A}, and L_{B} and the polymer simultaneously.

The index “c” is the degree of incorporation of Q, wherein “c” represents the average number of such monomer units in the composition, per polymer. “c” may be a finite number from about 3 to about 50.

M represents, in each instance, independently, H, a proton, an alkali ion; a pharmaceutically acceptable monovalent cation, or is absent.

The index “m” is the degree of incorporation of monomer units not in any other group, wherein “m” represents the average number of such monomer units in the composition, per polymer. “m” may be a finite number from about 50 to about 700.

In one embodiment, the polymer conjugate is described by formula (V):

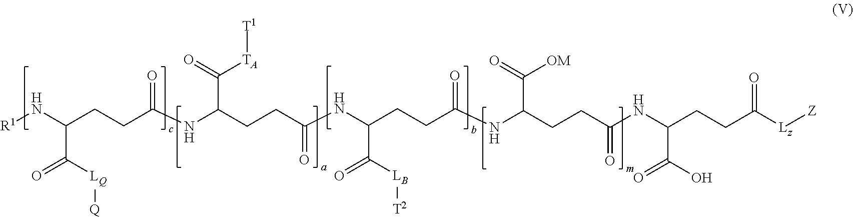

and metal ion complexes thereof.

The symbols in formula (V) that are in common with those in formula (IV) have the same meaning.

To the embodiment described by formula (IV), the embodiment of formula (V) further comprises a fluorophore moiety, Z, which is joined to the polymer at the C-terminal γ-carboxyl group, via linker group L_{Z}.

L_{Z} is any chemical moiety capable of linking the fluorophore moiety to the polymer backbone (here, the terminal monomer unit) via covalent bonds. In the event that Z can directly form a covalent bond with the polymer, then L_{Z} represents a bond. Otherwise, L_{Z} represents a bifunctional molecule having a first terminal site capable of forming a bond with a carboxyl group in a monomer unit, and a second terminal site capable of forming a bond with the suitable functional group in Z, wherein the first terminal site and the second terminal site are connected to one another through a chain of 3 to 20 atoms. The first terminal site may be an —O—, —S—, or an —NH—, thereby forming an ester, thioester, or amide link to the polymer. The second terminal site may be an —O—, —S—, —NH—, or an -acyl. The chain of 3 to 20 atoms may comprise a polyether, ether segments such as —O(CH₂CH₂)O— or an optionally substituted linear or branched hydrocarbon. The first terminal site of L_{Z} and the second terminal site of L_{Z} may be the same or different. For convenience, providing L_{Z} with the same first terminal site as one, some, or all of L_{A}, L_{B}, and L_{Q} may facilitate performing a coupling reaction between some or all of L_{Z}, L_{A} , L_{B}, and L_{Q} and the polymer simultaneously.

Though not expressly denoted in formula (V), the degree of incorporation of the fluorophore moiety at the terminal position might not be precisely 1. Instead, the degree of incorporation, may be about 0.8 to 1.2, as a result of varying reaction yields.

In one embodiment, the polymer conjugate is described by formula (VI):

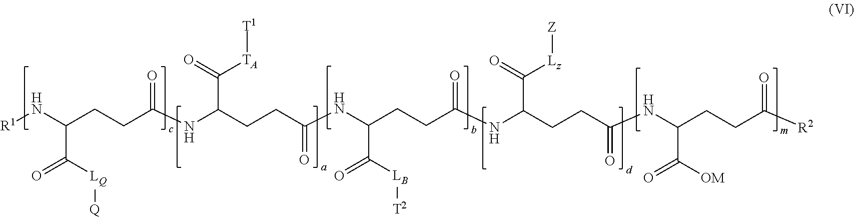

and metal ion complexes thereof.

The symbols in formula (VI) that are in common with those in formula (V) have the same meaning.

Compared to the embodiment described by formula (V), the embodiment of formula (VI) comprises the same components, however fluorophore moiety Z is joined to one of the monomer units at a pendent α-carboxyl group, via linker group L_{Z}.

The index “d” is the degree of incorporation of Z, wherein “d” represents the average number of such monomer units in the composition, per polymer. “c” may be a finite number that is approximately (greater than or less than) 1.

### B. Components of Polymer Conjugates

Polymer backbone. Polymers contemplated herein for use as the macromolecular structure to which the various moieties and ions are bound include biodegradable, non-immunogenic polymers that are safe for pharmaceutical use. The polymers comprise monomer units that provide a carboxylic acid functional group that may be used to conjugate functional moieties thereto or to interact with and bind cations, such as the metal ions.

In one embodiment, the polymers substantially comprise monomer units joined by peptide bonds. In further embodiments, the monomer units are selected from any form of glutamic acid. Forms of glutamic acid include the L isomer, the D isomer, or the DL racemate of glutamic acid. Any of these forms may be used, and two or more different forms may be used together in any proportion.

In another embodiment, glutamic acid monomer units may be joined in a peptide bond through either the α-carboxylic acid group or the γ-carboxylic acid group. In one embodiment, the same carboxylic acid group is used repeatedly in the polymer, to provide a polymer comprising uniform segments of α-polyglutamic acid (α-PGA) or γ-polyglutamic acid (γ-PGA). In further embodiments, the same connectivity is found throughout the polymer, that is, the polymer may be a homopolymer of α-PGA or γ-PGA. The various isomeric forms of α-PGA and γ-PGA may be synthetic or derived from natural sources. Whereas organisms usually only produce poly(amino acids) from the L isomer, certain bacterial enzymes that produce α-PGA or γ-PGA can produce polymers from either isomer or both isomers.

Polymers comprising glutamic acid monomer units, including those consist of α-PGA or γ-PGA, may be provided in various sizes and various polymer dispersities. The polymer molecular weight is generally at least about 5 kDa and at most about 100 kDa. In some embodiments, the polymer molecular weight is at least about 5 kDa, or least about 10 kDa, or at least about 20 kDa, or least about 30 kDa, or at least about 35 kDa, or at least about 40 kDa, or at least about 50 kDa. In some embodiments, the polymer molecular weight is at most about 100 kDa, or at most about 90 kDa, or at most about 80 kDa, or at most about 70 kDa, or at most about 60 kDa. An acceptable polymer molecular weight range may be selected from any of the above indicated polymer molecular weight values. In an embodiment, the polymer molecule weight is in the range of about 5 kDa to about 50 kDa. In an embodiment, the polymer molecule weight is in the range of about 50 kDa to about 100 kDa. In one embodiment, the polymer molecular weight is about 50 kDa. Polymer molecular weights are typically given as a number average molecular weight (Mₙ) based on a measurement by gel permeation chromatography (GPC). The above polymer masses are cited as Mₙ; other measurement techniques can be used to determine, e.g., a mass (weight) average molecular weight (M_{w}), and the specification for any given polymer can be converted among the various polymer mass representations.

Targeting Moieties. Targeting moieties contemplated include folate receptor ligands, such as folic acid, and analogs or derivatives thereof, and integrin-targeting peptides, such as RGD peptides and analogs or derivatives thereof.

Folate receptor protein is often expressed in many human tumors. Folates naturally have a high affinity for the folate receptors, and further, upon binding, the folate and the attached conjugate may be transported into the cell by endocytosis. In this way, a polymer conjugate comprising a folic acid moiety can target and accumulate at tumor cells and deliver the metal ion to the vicinity of and/or inside the tumor cells.

N⁵, N¹⁰-dimethyl tetrahydrofolate (DMTHF) is also known to have a high affinity for folate receptors. The preparation of DMTHF is described in Leamon, C. P. et al., Bioconjugate Chemistry 13, 1200-1210. Furthermore, there are two major isoforms of the folate receptor (FR), FR-α and FR-β, and DMTHF has been shown to have a higher affinity for FR-α over FR-β (Vaitilingam, B., et al., The Journal of Nuclear Medicine 53, 1127-1134.). This is beneficial for targeting tumor cells because FR-α is overexpressed in many malignant cell types, whereas FR-β is overexpressed on macrophages associated with inflammatory disease. Thus, folate-based targeting moieties may offer means to selectively bind to folate receptors expressed by tumor cells or macrophages.

Similarly, RGD peptides are known to bind strongly to α(V)β(3) integrins, which are expressed on tumoral endothelial cells as well as on some tumor cells. Thus, RGD conjugates may be used for targeting and delivering antitumor agents to the tumor site. Further information regarding the integrin family of cell surface receptors, their role in various pathologies, including cancer, inflammatory, or autoimmune disease, the natural ligands and analogs or mimetics that bind thereto, and conjugation methods have been reviewed recently (Wu, P-H., et al. (2019) Targeting integrins in cancer nanomedicine: applications in cancer diagnosis and therapy. Cancers 11, 1783).

With regard to conjugating a targeting moiety to the polymer backbone, folic acid has an exocyclic amine group that may be coupled with the free carboxylic acid group of a monomer (such as a glutamic acid monomer unit) to form an amide bond joining the two. The same exocyclic amine group as in folic acid is available in DMTHF for amide bond formation. RGD conjugates are also well-known in the art, and can also be similarly covalently joined to the free carboxylic acid group via, for example, the free α-amino group in RGD. Alternatively, either moiety may be conjugated to the polymer backbone via a linking group (e.g., L_{A} or L_{B}), such as, for example, polyethylene glycol amine. Examples of conjugation reactions between the γ-carbon carboxylate group of α-PGA and an amino group can be found in U.S. Pat. No. 9,636,411 to Bai et al. and with an amino and hydroxyl group can be found in US Pre-Grant Publication No. 2008/0279778 by Van et al. Examples of conjugation reactions to γ-PGA, including that of folic acid and citric acid, can be found in WO 2014/155142 (published Oct. 2, 2014). Other synthetic schemes are provided in the Examples below.

Ionophore Moieties. Ionophore moieties comprising an ionophore for the metal ion, are contemplated herein for joining to the polymer conjugate via a cleavable linker group, as a means for providing a ligand that may assist the metal ion in entering the intracellular compartment of a cell, in order to enhance the therapeutic activity of the metal ion. Exemplary ionophores include pyrithione and 1-hydroxypyridine-2-thione, and those of skill in the art can identify other suitable ionophores. These ionophores have the beneficial property that the sulfur moiety in the ionophore may be used to form a disulfide bond with a thiol group in an ionophore linking group (L_{Q}), wherein the ionophore will be masked and not bind with the metal ion until a later time, when the disulfide bond is cleaved, simultaneously releasing and unmasking the ionophore. In another embodiment, the ionophore binding site is not masked prior to being cleaved, and may interact with, including bind with, the metal ion both before and after the ionophore is cleaved from the polymer conjugate. Without being bound by theory, it is believed this occurs when a metal ion-polymer conjugate composition is take up by a cell, and the conditions therein favor the cleavage of the disulfide. As result, a metal ion-ionophore complex may form, and/or the ionophore may assist the metal ion to cross into intracellular compartments.

Label Moieties. Label moieties comprising a detectable label, such as, for example, a fluorophore, are contemplated herein for joining to the polymer conjugate as means to readily detect and/or quantify the polymer conjugate compositions. It is contemplated to be of particular use in investigative studies. Any standard fluorophore known to those skilled in the art may be used, provided the fluorophore structure includes a functional group that permits the fluorophore to be readily conjugated to the polymer, either directly or via a linking group (L_{Z}). Exemplary fluorophores include cyanine dyes, including Cy3, Cy3.5, Cy5, Cy5.5, and Cy7 dyes, as well as other analogs and derivatives of these dyes that are commercially available. The particular choice of dye may depend on the particular excitation and emission wavelength offered by each compound, or the availability of activated synthetic intermediates that are provided ready for conjugation reactions.

Metal ion. Metal ions contemplated herein for metal ion-polymer conjugate compositions include zinc(II) and cadmium(II) ions. Metal ion salts are used to prepare the compositions, e.g., zinc(II) salts (equivalently, Zn²⁺ salts), wherein the counterion (anion) may be any inorganic or organic anion suitable for use in the manufacture of a pharmaceutical product. Suitable anions are those that are tolerated by the human body, including those that are not toxic. Generally, the metal ion salt can be represented by the formulas M²⁺X²⁻ or M²⁺(X⁻)₂ or even M²⁺(X⁻)(Y⁻), where M²⁺ is Zn²⁺ or Cd²⁺ and X^{2-/1-} and Y^{2-/1-} are suitable anions. The anion may be selected from the group of anions that are a component of an FDA-approved pharmaceutical product. In some embodiments, the metal(11) salt is a pharmaceutically acceptable metal salt. Examples of zinc salts include zinc chloride, zinc sulfate, zinc citrate, zinc acetate, zinc picolinate, zinc gluconate, amino acid-zinc chelates, such as zinc glycinate, or other amino acids known and used in the art. Examples of cadmium salts include cadmium chloride, cadmium sulfate, and other salts as known and used in the art.

### C. Metal Ion-Polymer Conjugate Complexes

Metal ion-polymer conjugate complexes may be prepared by combining a metal ion with a polymer conjugate composition described herein. The amount of metal ion in the complexes according to the invention may be expressed as a mol ratio of the metal ion to monomer units (“MU”) or the weight ratio of the metal ion to the polymer conjugate. The mol ratio contemplated ranges from 1:2, 1:5, 1:10, 1:20, are contemplated, and even lower ratios are possible, but then the amount of PGA included in a dosage amount needed to deliver a suitable dose of the metal ion increases. An appropriate balance between the dosage amount and amount of non-zinc component can be determined by one of ordinary skill in the art. In one embodiment, the ratio is any value between about 1:2 and about 1:10 Zn:MU. In another embodiment, the ratio is any value between about 1:3 and about 1:6. In another embodiment, the ratio is about 1:4.5. In other embodiments, the metal ion is combined with the polymer conjugate at a particular weight ratio of the two components to form the metal ion-polymer conjugate composition. In one embodiment, the metal ion:polymer conjugate weight ratio is between about 1:5 to 1:20, and in one preferred embodiment, the weight ratio is about 1:10.

Metal ion-polymer conjugates complexes may be prepared by techniques known in the art for preparing metal ion complexes with a water-soluble polyelectrolyte, including the methods as disclosed in the Examples below. Such complexes may be prepared and stored as a solution, or lyophilized and stored as a solid. When prepared as a solution, the concentration of metal ion provided in a composition is generally in the range of about 1 μg/mL to about 100 mg/mL. This corresponds to a range of about 0.0001 wt % to about 10 wt % of the metal ion. The concentration may be at least about 10 μg/mL, or at least about 0.1 mg/mL, or at least about 1 mg/mL, or at least about 10 mg/mL, or at least about 50 mg/mL, or the range for the concentration may fall within any two of these exemplary concentrations. In one embodiment, the concentration may be in the range of about 100 μg/mL to about 5 mg/mL. In another embodiment, the concentration may be in the range of about 200 μg/mL about 2 mg/m L.

### D. Pharmaceutical Compositions

The metal ion-polymer conjugate compositions may be used to prepare pharmaceutical compositions or medicaments. In one embodiment, the pharmaceutical compositions or medicaments may be formulated as a liquid dosage form. Suitable liquid dosage forms include a solution, a suspension, a syrup, and an oral spray. Such solutions may be taken orally or administered by injection, such as intravenously, intradermally, intramuscularly, intrathecally, or subcutaneously, or directly into or in the vicinity of a tumor, whereas suspensions, syrups, and sprays are generally appropriate for oral administration. In another embodiment, the pharmaceutical compositions or medicaments may be formulated as a lyophilized form, to be reconstituted prior to administration as a liquid dosage form.

Preferably, the pharmaceutical compositions or medicaments further include one or more pharmaceutically-acceptable carriers, buffers, diluents, vehicles, excipients, or any combination thereof, suitable for administration to a subject, in particular a human patient, and suitable to render the pharmaceutical composition stable and efficacious for its intended purpose.

In some embodiments, the liquid dosage form is formulated for systemic delivery. In certain circumstances it may be desirable to administer the metal ion-polymer conjugate compositions parenterally, in suitably-formulated pharmaceutical vehicles, using standard delivery devices, including, without limitation, intravenously, subcutaneously, intramuscularly, intrathecally, intraperitoneally, transdermally, or topically. In other embodiments, liquid dosage forms may be administered by direct injection into one or more cells, tissues, or organs within or about the body, including a tumor mass, of a subject, in particular a human patient.

An embodiment of a liquid dosage form suitable for parenteral or oral delivery comprises a metal ion-polymer conjugate composition and water. In further embodiments, the liquid dosage form may further comprise a buffer and/or a salt, such as sodium chloride. When a buffering agent is included, a preferred buffer pH is in the range of about pH 4 to about pH 9. Also, in some embodiments of the liquid dosage form, the solution is isotonic with the fluid into which it is to be injected and of suitable pH. In any of the foregoing liquid dosage form embodiments, the solution may be prepared as a sterile solution. Such isotonic, buffered, sterile aqueous solutions are especially suitable for intravenous, intramuscular, subcutaneous, transdermal, subdermal, and/or intraperitoneal administration.

The pharmaceutical compositions disclosed herein may be formulated in a manner suitable for use in one or more pharmaceutically acceptable vehicles, including for example sterile aqueous media, buffers, diluents. For example, a given dosage of a metal ion-polymer conjugate composition may be dissolved in a particular volume of an isotonic solution (e.g., an isotonic saline solution), and then injected at the proposed site of administration, or further diluted in a vehicle suitable for intravenous infusion.

Liquid dosage forms may be packaged in vials, ampoules, cartridges, and prefilled syringes, and the like. In some embodiments, the liquid dosage form may be diluted before administration, and/or transferred from the package or delivery device to another delivery device, such as from a vial to a transfusion device. Lyophilized forms may be packaged in vials, cartridges, suitable syringe devices, and other suitable mixing systems that facilitate reconstituting the lyophilate to a liquid dosage form.

### E. Methods of Use

The pharmaceutical compositions described herein may be administered to provide a therapeutically effective amount of the metal ion-polymer conjugate composition to achieve the desired biological response in a subject. A therapeutically effective amount means that the amount of metal ion delivered to the patient in need of treatment, through the combined effects of the metal ion, the polymer conjugate, including the moieties conjugated thereto or released therefrom, and/or the delivery efficiency of the dosage form, and the like, will achieve the desired biological response. The therapeutically effective amount may also differ according to the indication being treated and the condition of the subject.

The desired biological response include the prevention of the onset or development of a tumor or cancer, the partial or total prevention, delay, or inhibition of the progression of a tumor or cancer, or the prevention, delay, or inhibition of the recurrence of a tumor or cancer in the subject, such as a mammal, particularly in a human (also may be referred to as a patient). Clinical benefits of the treatment methods can be assessed by objective response rate, tumor size, duration of response, time to treatment failure, progression free survival, and other primary and secondary endpoints assessed in clinical use.

All tumor types or macrophage-mediated inflammatory pathologies that are susceptible to PARP-mediated necrosis are contemplated to be indications that can be treated according to the methods of treatment disclosed herein. The various examples demonstrate the efficacy of treatments according to embodiments of the disclosed methods using embodiments of the disclosed compositions and pharmaceutical formulations. The results demonstrate effective treatments of mouse cancer cells in vitro, human cancer cells ex vivo, and in vivo treatment effects in syngenic murine cancer models.

Achieving a therapeutically effective amount will depend on the formulation's characteristics, any will vary by gender, age, condition, and genetic makeup of each individual. In the case of zinc metal complexes, individuals with inadequate zinc due to, for example, genetic causes or other causes of malabsorption or severe dietary restriction may require a different amount for therapeutic effect compared to those with generally adequate levels of zinc.

The subject is generally administered an amount of metal ion from about 0.1 mg/kg/day up to about 6 mg/kg/day. In some embodiments, the amount of metal ion, e.g., zinc, administered is from about 1.0 mg/kg/day to about 4 mg/kg/day. Multiple dosage forms may be taken together or separately in the day. Treatment generally continues until the desired therapeutic effect is achieved. Low dosage levels of the compositions and formulations described herein may also be continued as a treatment according to an embodiment of the invention if a tumor regresses or is inhibited, for the purpose of preventing, delaying, or inhibiting its recurrence, or used as a preventative treatment.

### EXAMPLES

The invention is further defined in the following Examples. It should be understood that the Examples are given by way of illustration only, wherein the embodiments are intended to demonstrate aspects of the invention in a non-limiting manner. From the description and the Examples disclosed herein, one skilled in the art can ascertain the essential characteristics of the disclosure, and without departing from the spirit and scope thereof, can make various changes and modifications to adapt the disclosure and still obtain a similar product or result. As a result, the disclosure is not limited by the illustrative examples set forth herein.

The following abbreviations are used below: ACN is acetonitrile; DCC is N,N′-dicyclohexylcarbodiimide; DIC is N,N′-diisopropylcarbodiimide; DIEA is N,N-diisopropylethylamine; DMSO is dimethyl sulfoxide; EDC is 1-ethyul-3-(3-dimethylaminopropyl)carbodiimide; equiv. is equivalent; HBTU is 2-(1H-benzotriazol-1-yl)-1,1,3,3-tetramethyluronium hexafluorophosphate; HOBt is hydroxybenzotriazole; MTBE is methyl tent-butyl ether; NHS is N-hydroxysuccinimide; NLT is not less than; NMT is not more than; TFA is trifluoroacetic acid.

### A. Preparation of Polymer Conjugates and Metal Ion Complexes Thereof

### Example 1: Preparation of Folate-NH-PEG₄-NH₂ Side Chain

A folate side chain, folate-NH-PEG₄-NH₂, was prepared according to the synthetic scheme shown below:

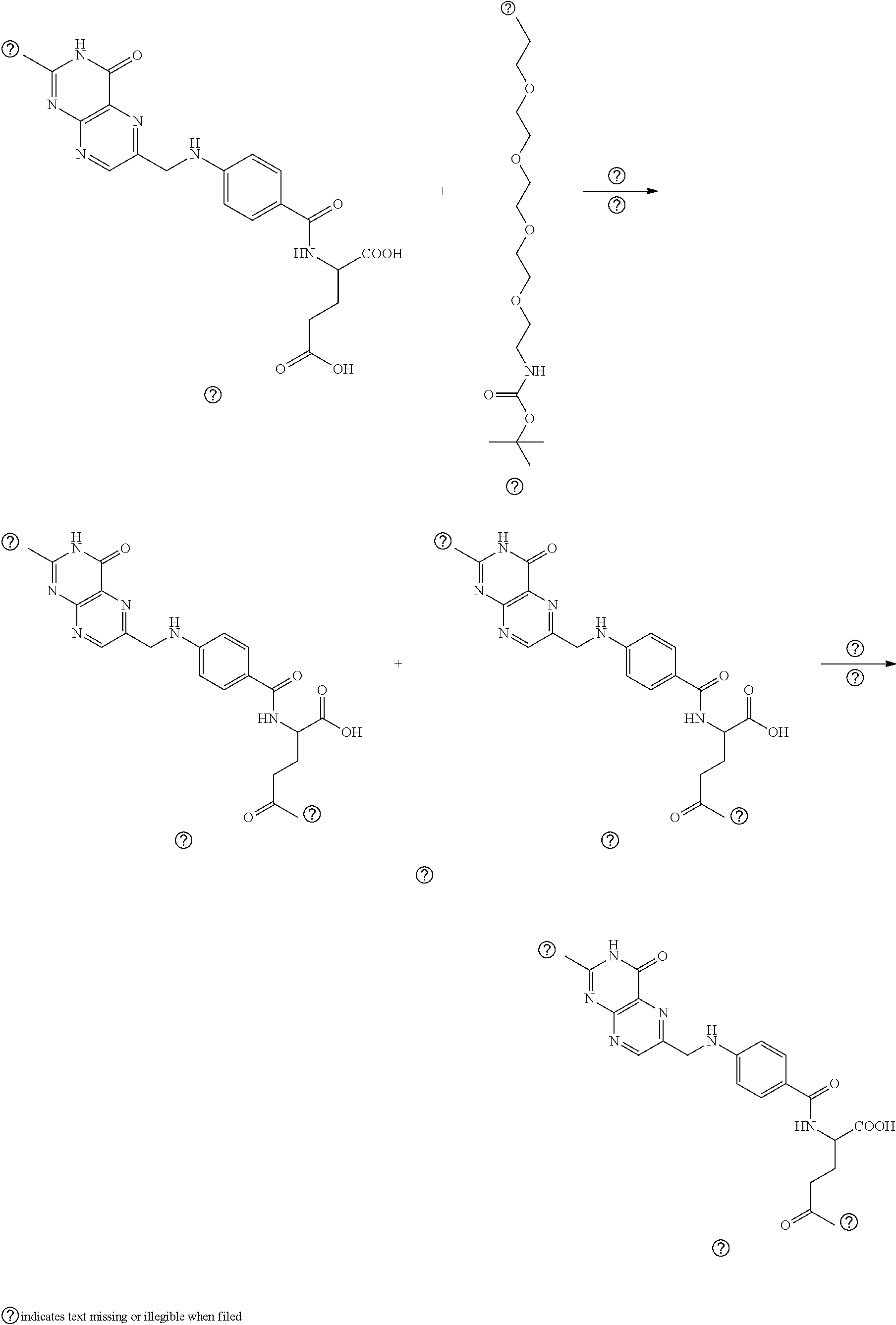 [SMILES: Cc1nc2ncc(CNc3ccc(C(=O)NC(CCC(=O)O)C(=O)O)cc3)nc2c(=O)[nH]1 ; CCCOCCOCCOCCOCCNC(=O)OC(C)(C)C ; CC(=O)CCC(NC(=O)c1ccc(NCc2cnc3nc(C)[nH]c(=O)c3n2)cc1)C(=O)O ; CC(=O)CCC(NC(=O)c1ccc(NCc2cnc3nc(C)[nH]c(=O)c3n2)cc1)C(=O)O ; CC(=O)CCC(NC(=O)c1ccc(NCc2cnc3nc(C)[nH]c(=O)c3n2)cc1)C(=O)O]

### A. Synthesis of Folate-NH-PEG₄-NH-Boc

A 10 g scale synthesis was performed by dissolving folic acid (10 g, 1 equiv.) in DMSO (350 mL)/pyridine (150 mL) solution charged with DIC (5.72 g, 2 equiv.) and stirred for 0.5 hr at 25° C. Boc-NH-PEG₄-NH₂ (9.15 g, 1.2 equiv.) dissolved in DMSO (10 mL) was added and stirred for three days. On the fourth day, an additional charge of DIC (1.43 g, 0.5 equiv.) was added and stirring was continued for one day. At the end of the reaction time the γ-isomer α-isomer ratio was found to be 95:5. Pyridine was removed by rotary evaporation at 40° C. The remaining solution was poured into MTBE (4 vol equiv.) then the upper layer was removed. MTBE (2 vol. equiv.) was added to slurry the brown sticky compound twice, and each time the upper layer was removed. MTBE (1 vol. equiv.) was added to slurry the orange sticky compound, and then the upper layer was removed. As a result of repeating the slurrying process several times, the sticky brown crude product turned to an orange/yellow solid. After filtration and drying the solid under N₂, the crude Boc-folate side chain were obtained as orange to yellow powder (15.1 g, 88% crude yield). The product was confirmed by proton NMR (d⁶-DMSO). Other preparations at 20 g scale yielded similar results.

B. Synthesis of Folate-NH-PEG₄-NH₂

Folate-NH-PEG₄-NH-Boc (10.0 g, 1.0 equiv.) was treated with trifluoroacetic acid (100 mL) at room temperature, and the progress of the reaction was monitored by HPLC. TFA was removed by concentration under reduced pressure at 40° C., and a brown viscous oil was obtained. This oil was purified by ion exchange using a DEAE Sephadex A-25 column (200 g; pretreated in 0.1 M potassium tetraborate (K₂B₄O₇) tetrahydrate and changed to H₂O phase). The oil was diluted with water (5 mL), added to the column, and washed with one volume of H₂O and eluted with 50 mM ammonium bicarbonate solution. Fractions containing the product were collected (estimated product yield: 81%), combined with another similarly prepared batch to provide ˜2.4 g of the crude product in ˜4.2 L ammonium bicarbonate solution. This solution was further purified using LiChroprep RP-18 (40-63 μm) (100 g). After loading the material onto the column, the column was washed with H₂O, acidified to pH 2 by washing with 5 volumes of 0.1% TFA/H₂O, and the product was eluted with 9:1H₂O(0.1% TFA)/ACN. Appropriate fractions were collected and concentrated under reduced pressure at 35° C. to ˜200 mL, which was lyophilized to yield folate-NH-PEG₄-NH₂ (1.988 g, 70% yield, 90.8% purity) as a yellow fluffy solid.

The product was confirmed by proton NMR (d⁶-DMSO) and ESI mass spectroscopy (m/z: 658.29 [M-H]⁺).

### Example 2: Preparation of cRGDfk-(O)C-PEG₄-NH₂ Side Chain

A cyclic-RGD side chain, cRGDfk-(O)C-PEG₄-NH₂, was prepared according to the synthetic scheme shown below:

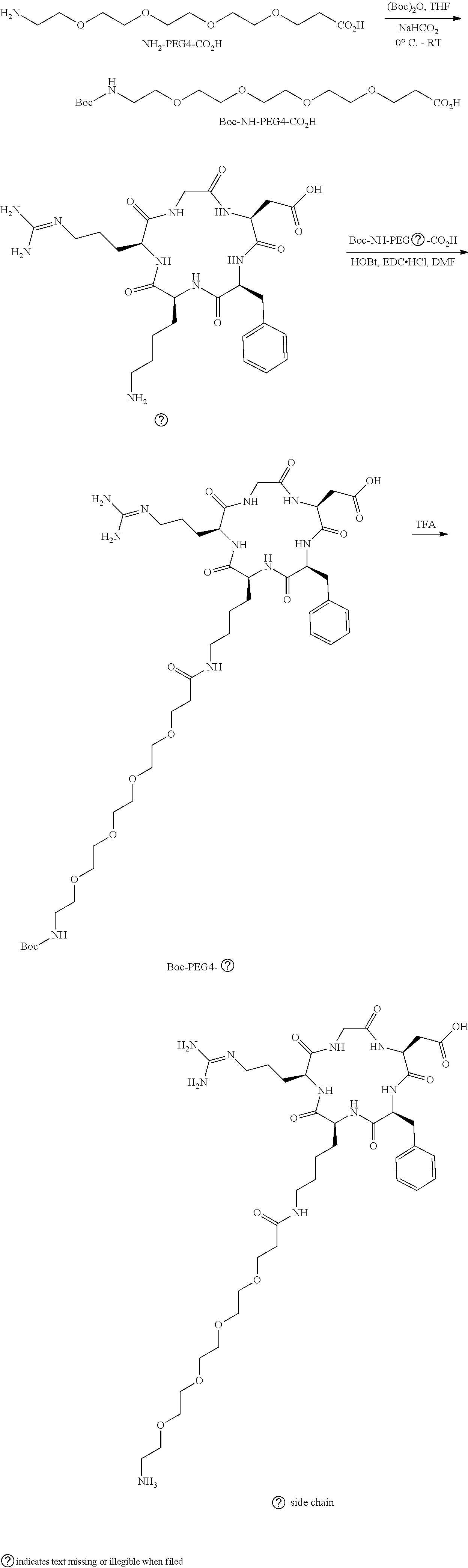 [SMILES: NCCOCCOCCOCCOCCC(=O)O ; CC(C)(C)OC(=O)NCCOCCOCCOCCOCCC(=O)O ; NCCCC[C@@H]1NC(=O)[C@H](Cc2ccccc2)NC(=O)[C@H](CC(=O)O)NC(=O)CNC(=O)[C@H](CCCN=C(N)N)NC1=O ; CC(C)(C)OC(=O)NCCOCCOCCOCCOCCC(=O)NCCCC[C@@H]1NC(=O)[C@H](Cc2ccccc2)NC(=O)[C@H](CC(=O)O)NC(=O)CNC(=O)[C@H](CCCN=C(N)N)NC1=O ; NCCOCCOCCOCCOCCC(=O)NCCCC[C@@H]1NC(=O)[C@H](Cc2ccccc2)NC(=O)[C@H](CC(=O)O)NC(=O)CNC(=O)[C@H](CCCN=C(N)N)NC1=O]

A. Synthesis of Boc-NH-PEG₄-CO₂H

To a solution of H₂O (50 mL), NH₂-PEG₄-CO₂H (5 g, 1 equiv.) and NaHCO₃ (4.74 g, 3 equiv.) were added. The mixture was cooled to 0-5° C., (Boc)₂O (4.94 g, 1.2 equiv.) in THF (50 mL) was added, and the resulting mixture was stirred at 0-5° C. for 1 hr. The mixture was warmed to room temperature and stirred overnight. The turbid solution was extracted with MTBE (80 mL×3). The aqueous layer was acidified to pH 4 by addition of 42% citric acid at NMT 10° C., and then extracted with CH₂C₁₂ (80 mL×3). The combined organic phase was dried over Na₂SO₄ and evaporated to dryness to provide Boc-NH-PEG₄-CO₂H (5.1 g, 74% yield) as colorless oil. The product was confirmed by proton NMR.

### B. Synthesis of Boc-NH-PEG₄-cRGDfk

Boc-NH-PEG₄-CO₂H (1300 mg, 1 equiv.) was dissolved in DMF (120 mL) at room temperature and then EDC-HCl (700 mg, 1 equiv.), NHS (430 mg, 1 equiv.) were added for activation. After 1 hr, cRGDfK (TFA counterion) (3 g, 1 equiv.) and DIPEA (870 uL, 1.2 equiv.) in DMF (45 mL) were added into the activated Boc-NH-PEG₄-CO₂H solution, stirred overnight, and then added into MTBE (3 L) to precipitate the product. After filtration and washing the filtrate with 80 mL MTBE, the crude product Boc-NH-PEG₄-cRGDfK (3.26 g, 80% yield) was obtained as white solid. The crude product (2.2 g) was further purified using C-18 gel (20 μm) (Diasogel Grade: SP-120-20-ODS-BP) by loading the crude product dissolved in water (230 mL), and eluting, using 0.1% HCl (aqueous) and 0.1% HCl in ACN as mobile phases A and B, by ramping the fraction of B from 0%, to 15%, and then to 25% in 5% increments. Appropriate fractions were collected, combined, and the solvent removed to yield the product Boc-NH-PEG₄-cRGDfK (0.6 g).

C. Synthesis of cRGDfk-PEG₄-NH₂

Boc-NH-PEG₄-cRGDfK (0.6 g) was deprotected by treating it with TFA (2 mL) at room temperature for 15 minutes, and the TFA was removed. The crude product was taken up in water (80 mL) and purified using C-18 gel as before for the Boc-protected precursor, except that the column was eluted by ramping the fraction of mobile phase B from 0% to 10% in 5% increments. Appropriate fractions were collected, combined, and the solvent removed to yield the product cRGDfK-PEG₄-NH₂ (0.22 g). The product was confirmed by proton NMR and ESI mass spectroscopy (m/z: 849.44 [M-H]⁺).

### Example 3: Preparation of Pyrithione-S-PEG₃-NH₂ Side Chain

An ionophore side chain, Pyr-S-PEG₃-NH₂, was prepared according to the synthetic scheme shown below:

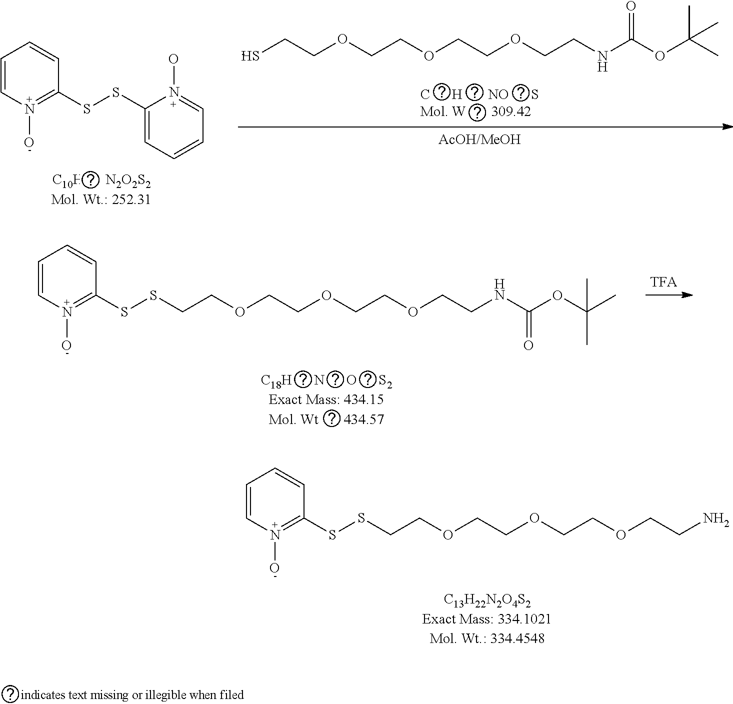

A. Synthesis of Pyr-S-PEG₃-NH-Boc Dipyrithione (8 g, 5 equiv.) and Boc-N-amido-PEG₃-thiol (2 g, 1 equiv.) were dissolved in a methanol (400 mL)/acetic acid (250 mL) solution and stirred for 24 hr at room temperature. After reaction completion, the reaction mixture was evaporated to dryness by rotary evaporation at 40° C. to obtain a white solid. Methanol was added to the solid and the resulting mixture was filtered. The cake was confirmed to be dipyrithione. The concentrated filtrate was added into 100 mL MTBE, and the resulting white precipitate (dipyrithione) was removed by filtration. The MTBE filtrate was determined to contain pyrithione and the target product. Pyrithione-PEG₃-NH-Boc was separated and purified using 40 g LiChroprep RP-18 (40-63 μm), using water and ACN as the mobile phases, and ramping ACN from 10% to 30% in 5% increments. Appropriate fractions were collected, combined, and the solvent removed to provide pyrithione-PEG₃-NH-Boc (2.04 g, 97.6% purity) as a colorless oil. The product was confirmed by ESI mass spectroscopy (m/z 457.1 [M+Na]⁺).
B. Synthesis of Pyr-S-PEG₃-NH₂

Pyrithione-S-PEG₃-NH-Boc (1.8 g) was deprotected by treating it with TFA (15 mL) at room temperature for 12 minutes, and the TFA was removed by rotary evaporation at 40° C. The crude product was taken up in water (6 mL) and lyophilized to provide the crude product pyrithione-S-PEG₃-NH₂ (3.0 g, 97% pure) as a clear, slightly black oil. The product was confirmed by proton NMR and ESI mass spectroscopy (m/z: 355.1 [M+H]⁺).

### Example 4: Preparation of Cy5.5-NH—(CH₂)₆—NH₂ Side Chain

A Cy5.5 side chain, Cy5.5-NH—(CH₂)₆—NH₂, was prepared according to the synthetic scheme shown below:

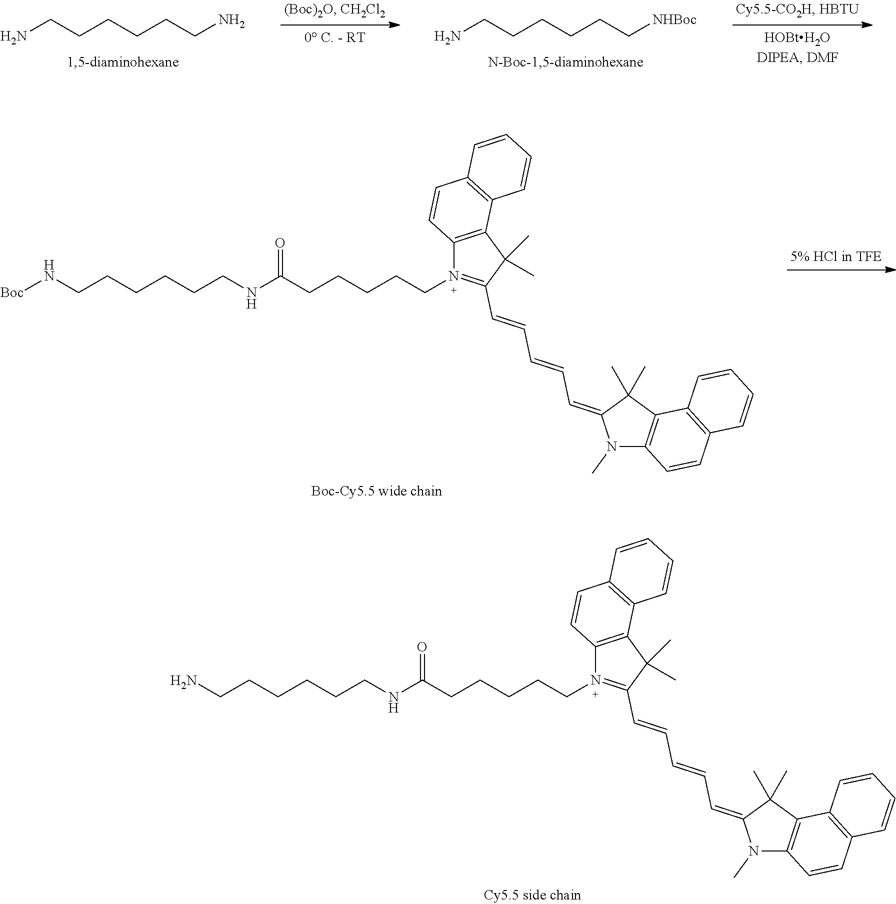

A. Synthesis of N-Boc-1,6-diaminohexane

To a solution of 1,6-diaminohexane (27.8 g, 5 equiv.) in CH₂Cl₂ (200 mL) was added a solution of Boc anhydride (10.45 g, 1 equiv.) in CH₂Cl₂ (60 mL) dropwise at NMT 5° C. The mixture was stirred at NMT 5° C. for 50 min and then stirred at room temperature overnight. The mixture was evaporated to dryness under vacuum at 85° C. The residue was washed with 120 mL of 15% Na₂CO₃ solution. After removing the H₂O layer, 30 mL of DCM was added, the phases separated, and the DCM layer was extracted with 52 mL of 15% Na₂CO₃ solution, and washed with 50 mL of H₂O four times to remove residual 1,6-diaminohexane. The organic layer was evaporated to dryness to afford crude N-Boc-1,6-hexyldiamine (7.21 g) as a slightly yellow oil in 70% crude yield. The product was confirmed by proton NMR. The crude product (4 mL) was dissolved in 10 mL DCM, dried over Na₂SO₄, and then evaporated to dryness under vacuum at 40° C. to obtain the desired product.

### B. Synthesis of Cy5.5-NH—(CH2)6—NH-Boc

Cy5.5-CO₂H (2.0 g, 1 equiv.), N-Boc-1,6-diaminohexane hydrochloride (816 mg, 1 equiv.), HBTU (3.68 g mg, 3 equiv.), HOBt hydrate (1.31 g, 3 equiv.), and DIEA (8.4 mL, 15 equiv.) were dissolved in 40 mL DMF. The mixture was stirred at room temperature for NLT 2.5 hr. After reaction completion as monitored by TLC, DCM (25 mL) was added to the mixture and extracted with H₂O (25 mL×4). The organic layer was dried over Na₂SO₄, filtered, and evaporated to dryness. The residue was purified by silica gel column chromatography (DCM/MeOH=9/1) to obtain the Boc-protected Cy5.5 side chain (2.95 g, 112% yield (residual solvent accounting for the overage)). The product was confirmed by proton NMR (CDCl₃).

C. Synthesis of Cy5.5-NH—(CH₂)₆—NH₂

Boc-protected Cy5.5 side chain (2.95 g, 1 equiv.) in a solution of 5% HCl (37%) in 2,2,2,-trifluoroethanol (94 mL) was stirred in the dark for 3 hr at room temperature. After reaction completion as monitored by TLC, the solvent was evaporated to dryness to obtain crude Cy5.5 side chain (3.44 g, 90% purity by HPLC). The crude product was purified by LiChroprep RP-18 (40-63 μm) using 0.1% HCl (aqueous) and 0.1% HCl in ACN as mobile phases A and B. The crude product was dissolved in 10% (v/v) ACN with 0.1% HCl, applied to the column, and eluted by ramping the fraction of B from 10% to 60% in 10% increments. Appropriate fractions were collected, combined, and lyophilized to yield Cy5.5-NH—(CH₂)₆—NH₂ (1.33 g, 51% yield, 99% purity) as a deep blue solid. The product was confirmed by proton NMR (methanol-d4) and ESI mass spectroscopy (m/z: 681.45 [M⁺]).

### Example 5: Preparation of γ-Polyglutamic Acid (γ-PGA-H)

The free acid form of γ-polyglutamic acid (γ-PGA-H) was prepared by acidifying a solution of the sodium salt of γ-PGA and using n-propanol to aid in precipitating the free acid.

γ-PGA-Na⁺ (100 g) having Mw ˜32 kDa, was dissolved in water (200 mL) and the solution was acidified to about pH 3 by adding 6N HCl (˜65 mL) at LT 10° C. The acidified solution was added into 99.5% EtOH/n-propanol (1:3 v/v) solution (1200 mL) over 5 minutes, and then stirred for 10 minutes, resulting in a white sticky precipitate. The solution was poured out, leaving an oily precipitate. Water (200 mL) was added to dissolve the precipitate, and the resulting solution was separated to two portions for further work up. Each portion was acidified by adjusting to ˜pH 2 using 6 N HCl over about 20-25 minutes, while keeping the solution under 10° C. Each portion was then added into 99.5% EtOH (1600 mL) over 5 minutes at room temperature, to obtain a white fluffy precipitate. After filtration and drying, γ-PGA-H (61.0 g) was obtained as a white solid. The γ-PGA-H was soluble in DMSO and H₂O.

### Example 6: Preparation of C010D-Zn

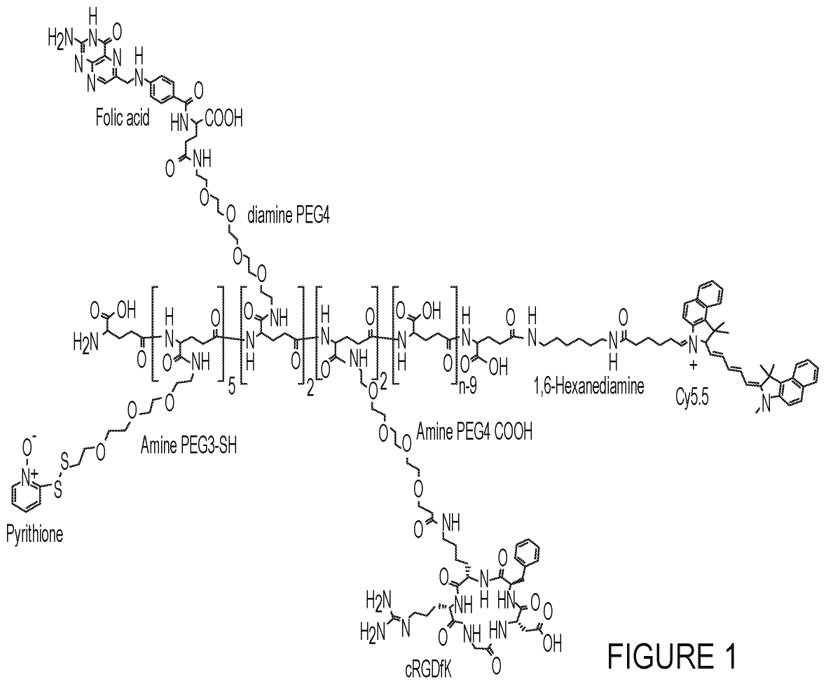

The polymer conjugate C010D has the nominal structure shown in FIG. 1.

The polypeptide polymer γ-polyglutamic acid (γ-PGA-H), and the four side chains bearing pyrithione, folate, cRGDfk, and Cy5.5 moieties, respectively, were prepared as described in Examples 1-5, and used as follows to prepare C010D. γ-PGA-H (0.76 g, 1 equiv.) was dissolved in DMSO (50 mL), and EDC·HCl (74 mg, 23 equiv.) and NHS (45 mg, 23 equiv.) were added, and the solution was stirred for 45 min. at room temperature. Each side chain was combined with 6 equiv DIPEA (as against the side chain) in DMSO (1 mL). The nominal ratio of each side chain to γ-PGA-H was: pyrithione: 5 equiv.; folate: 2 equiv.; cRGDfk: 2 equiv.; Cy5.5: 1 equiv. The DMSO solutions with each side chain were added to the activated γ-PGA-H, and the solution was stirred for 21 hr while monitoring consumption of the side chains by HPLC. Upon completion, the reaction was taken up in water (200 mL), the pH adjusted from 4.2 to 8.84 with 6N NaOH/6N HCl, filtered through 0.22 μm PVDF membrane, and then purified, including DMSO removal, by tangential flow filtration using a Millipore Pellicon® 3 cassette (0.11 m ², 5 kD membrane) followed by 6 times of diafiltration (adding 2000 mL H₂O each time), and providing a final volume of 220 mL. Impurities were removed by filtration again, and the filtrate was lyophilized to yield C010D polymer conjugate (0.874 g) as a blue solid. The degree of incorporation of each side chain was determined by proton NMR (D₂O/DMSO-d₆). Per γ-PGA polymer, the side chains pyrithione : folate : cRGDfk : Cy5.5 were present at the ratio of 3.96: 1.84: 1.71 : 1.09.

C010D-Zn was prepared by mixing C010D (279 mg, assayed amount of polymer conjugate: 71.65%) with zinc sulfate heptahydrate (88 mg; 0.1 equiv, per polymer conjugate) in 30 mM HEPES (pH 7) aqueous solution (20 mL). In this solution, the weight/weight ratio between the zinc ion and the γ-PGA backbone of C010D was 1:10, and it was used in some of the following examples of biological tests as is, unless otherwise noted.

### Example 7: Preparation of C010DS-Zn

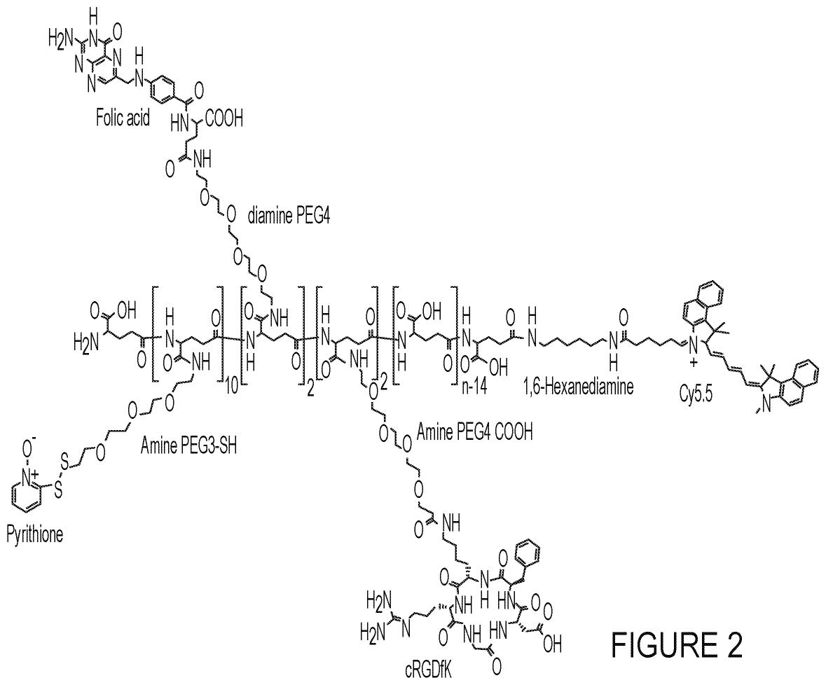

The polymer conjugate CO1ODS has the nominal structure shown in FIG. 2.

The polypeptide polymer γ-polyglutamic acid (γ-PGA-H), and the four side chains bearing pyrithione, folate, cRGDfk, and Cy5.5 moieties, respectively, were prepared as described in Examples 1-5, and used as follows to prepare C010DS. γ-PGA-H (0.76 g, 1 equiv.) was dissolved in DMSO (50 mL), and EDC·HCl (113 mg, 35 equiv.) and NHS (68 mg, 35 equiv.) were added, and the solution was stirred for 45 min. at room temperature. Each side chain was combined with 6 equiv DIPEA (as against the side chain) in DMSO (1 mL). The nominal ratio of each side chain to γ-PGA-H was: pyrithione: 10 equiv.; folate: 2 equiv.; cRGDfk: 2 equiv.; Cy5.5: 1 equiv. The DMSO solutions with each side chain were added to the activated γ-PGA-H, and the solution was stirred for 21 hr while monitoring consumption of the side chains by HPLC. Upon completion, the reaction was worked up and purified using the same procedure described in Example 6 to yield C010DS polymer conjugate (0.9 g) as a blue solid. The degree of incorporation of each side chain was determined by proton NMR (D₂O/DMSO-d₆). Per γ-PGA polymer, the side chains pyrithione : folate : cRGDfk : Cy5.5 were present at the ratio of 7.94: 1.82: 1.70: 0.96.

C010DS-Zn was prepared by mixing C010DS (309 mg, assayed amount of polymer conjugate: 64.77%) with zinc sulfate heptahydrate (88 mg; 0.1 equiv, per polymer conjugate) in 30 mM HEPES (pH 7) aqueous solution (20 mL). In this solution, the weight/weight ratio between the zinc ion and the γ-PGA backbone of C010D was 1:10, and it was used in some of the following examples of biological tests as is, unless otherwise noted.

### Example 8: Preparation of 010DS(P50)-Zn

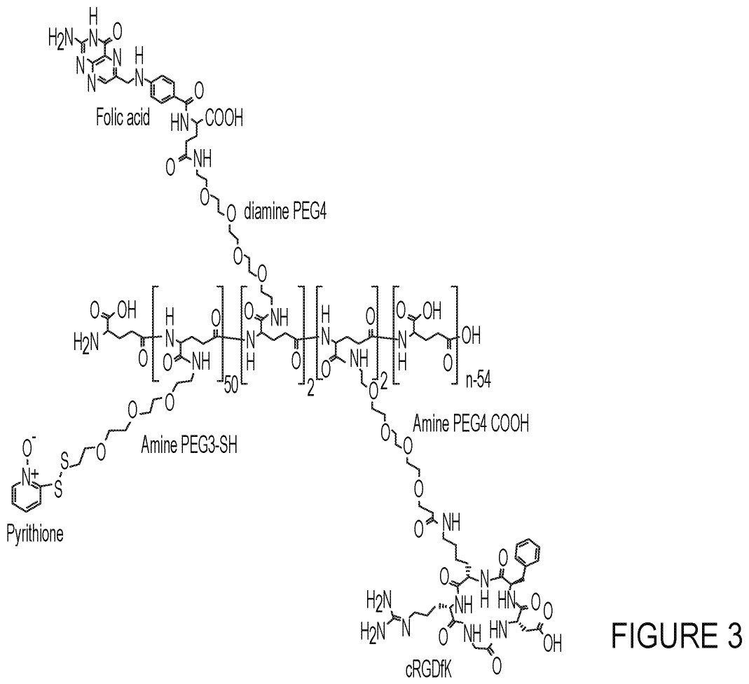

The polymer conjugate 010DS(P50) has the nominal structure shown in FIG. 3.

The polypeptide polymer γ-polyglutamic acid (γ-PGA-H), and the three side chains bearing pyrithione, folate, and cRGDfk moieties, respectively, were prepared as described in Examples 1-3 and 5, and used as follows to prepare 010DS. γ-PGA-H (37.1 g, 1 equiv.) was dissolved in DMSO (2250 mL), and DIC (6.77 g, 65 equiv.), NHS (6.17 g, 65 equiv.), and NaOH (2.14 g, 65 equiv.) were added, and the solution was stirred for 60 min. at room temperature, under N₂. Each side chain was dissolved in DMSO (45 mL). The nominal ratio of each side chain to γ-PGA-H was: pyrithione: 25 equiv.; folate: 2 equiv.; cRGDfk: 2 equiv. The DMSO solutions with each side chain were added to the activated γ-PGA-H, and the solution was stirred for 17.5 hr while monitoring consumption of the side chains by HPLC. Thereafter, a second stage to add an additional 25 equiv. of pyrithione side chain was commenced. At 18 hr, to this reaction mixture was added DIC (7.8 g, 75 equiv. against γ-PGA-H), at 20 hr, pyrithione side chain (25 equiv.) in 45 mL DMSO, and at 20.5 hr, NaOH (181 mg, 5.5 equiv.). At 23 hr, another NaOH (181 mg, 5.5 equiv.) was added, and at 24 hr, NHS (7.12 g, 75 equiv.) was added. As of 24.4 hr the pyrithione side chains remained unreacted, but thereafter the reaction commenced. At 46 hr, an additional NaOH (350 mg, 10.6 equiv.) was added, and at 47.5 hr the pyrithione side chain conjugation reaction had progressed 37%. After 112.5 hr, the side chain conjugation reaction has progressed 97%, and the reaction was stopped. Thereafter, the reaction solution was added into 9280 mL water, the pH was adjusted to 7.0 by addition of 6N NaOH. The solution was purified using the same procedure described in Example 6, scaled appropriately, and finally lyophilized to yield 010DS polymer conjugate (46.6 g) as a yellow solid. The degree of incorporation of each side chain was determined by proton NMR (D₂O/DMSO-d₆). Per γ-PGA polymer, the side chains pyrithione:folate:cRGDfk were present at the ratio of 47.10:1.58:1.91. The molecular weight (Mw) was determined to be 40,085. In addition, MP was 39,698, Mn was 25,872, Mz was 62,852, and the polydispersity index was 1.55.

010DS(P50)-Zn was prepared by mixing 010DS(P50) (36.7 g, assayed amount of polymer conjugate: 68.13%) with zinc sulfate heptahydrate (11 g; 0.1 equiv, per polymer conjugate) in water (184 mL). This solution was lyophilized to yield 010DS(P50)-Zn (40.1 g) as a yellow solid. The weight/weight ratio between the zinc ion and the γ-PGA backbone of 010DS(P50) was 1:10.

### Example 9: Preparation of C005D-Zn (Control)

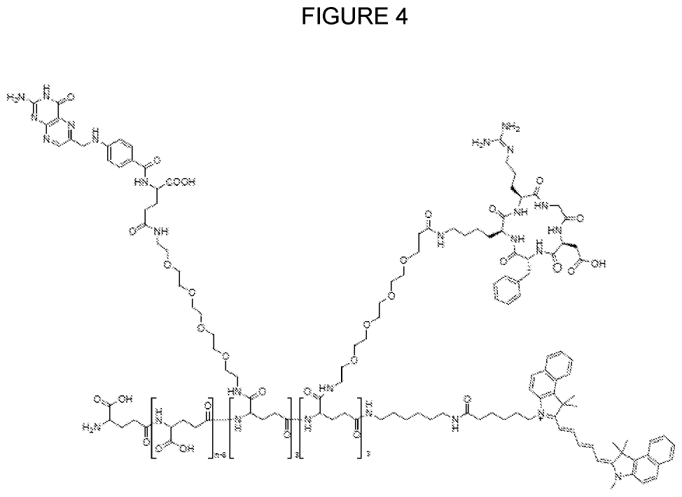

The polymer conjugate C005D has the nominal structure shown in FIG. 4.

The polypeptide polymer γ-polyglutamic acid (γ-PGA-H), and the three side chains bearing folate, cRGDfk, and Cy5.5 moieties, respectively, were prepared as described in Examples 1-2 and 4-5, and used as follows to prepare C005D. γ-PGA-H (50 g, 1 equiv.) was dissolved in DMSO (350 mL), and EDC·HCl (3.2 g, 15 equiv.) and NHS (1.92 g, 15 equiv.) were added, and the solution was stirred for 60 min. at room temperature. Each side chain was combined with 6 equiv DIPEA (as against the side chain) in DMSO (50 mL). The nominal ratio of each side chain to γ-PGA-H was: folate: 3 equiv.; cRGDfk: 3 equiv.; Cy5.5: 1 equiv. The DMSO solutions with each side chain were added to the activated γ-PGA-H, and the solution was stirred for 30 hr while monitoring consumption of the side chains by HPLC. Upon completion, water (100 mL) was added to the DMSO reaction, and all solvent was removed by lyophilization to yield the crude product C005D (130.34 g). worked up and purified using the same procedure described in Example 6 to yield C010DS polymer conjugate (42.56 g) as a blue solid. The degree of incorporation of each side chain was determined by proton NMR (D₂O/DMSO-d₆). Per γ-PGA polymer, the side chains folate:cRGDfk:Cy5.5 were present at the ratio of 3.25:3.14:0.79.

C005D-Zn was prepared by mixing C005D (10 g, assayed amount of polymer conjugate: 79.1%) with an aqueous solution (pre-filtered by 0.22 μm PVDF filter) of zinc sulfate heptahydrate (3.48 g; 0.1 equiv, per polymer conjugate) in water (15 mL). The pH of the solution was adjusted to 6.37 using 1 N NaOH (3 mL) and 6N HCl (350 μL). The solution was removed by lyophilization, to provide C005D (11.89 g) as a blue solid.

The solid was dissolved in 30 mM HEPES (pH 7) aqueous solution for use in some of the following examples for biological testing as a control, against some embodiments of the therapeutically-active metal ion polymer conjugate compositions according to the subject invention. In this solution, the weight/weight ratio between the zinc ion and the γ-PGA backbone of C005D was 1:10, unless otherwise noted.

### B. Biological Testing of the Polymer Conjugate-Metal Ion Complexes

### Example 10: In Vitro Cytotoxicity Test

The in vitro cytotoxicity of C010DS-Zn (prepared according to Example 7) and relevant control groups, including zinc sulfate, zinc sulfate-sodium Pyr mixture at 7 Zn²⁺ to 1 Pyr molar ratio (7Zn-1Pyr) (to match the C010DS-Zn payload), and C005D-Zn (a positive control compound with identical structure and zinc content minus the Pyr-sidechain modifications)(prepared according to Example 9) were tested.

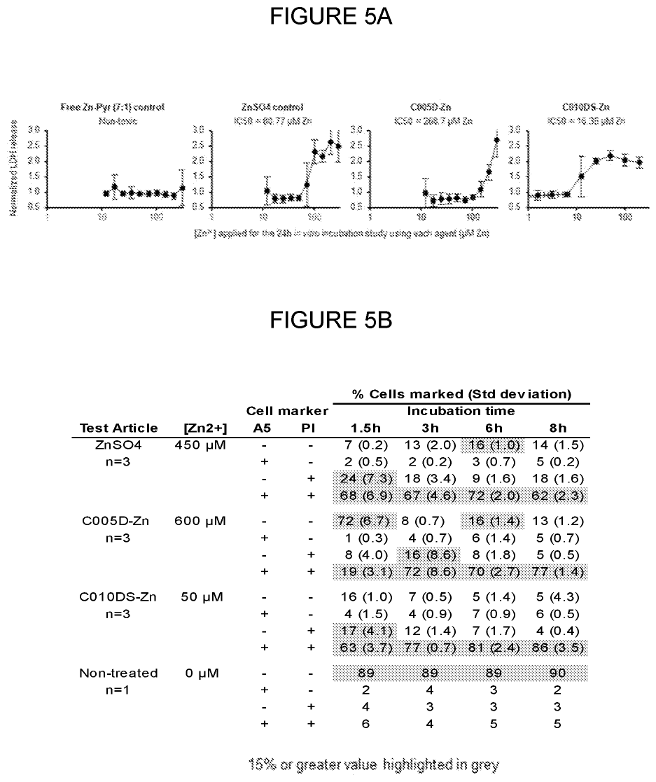

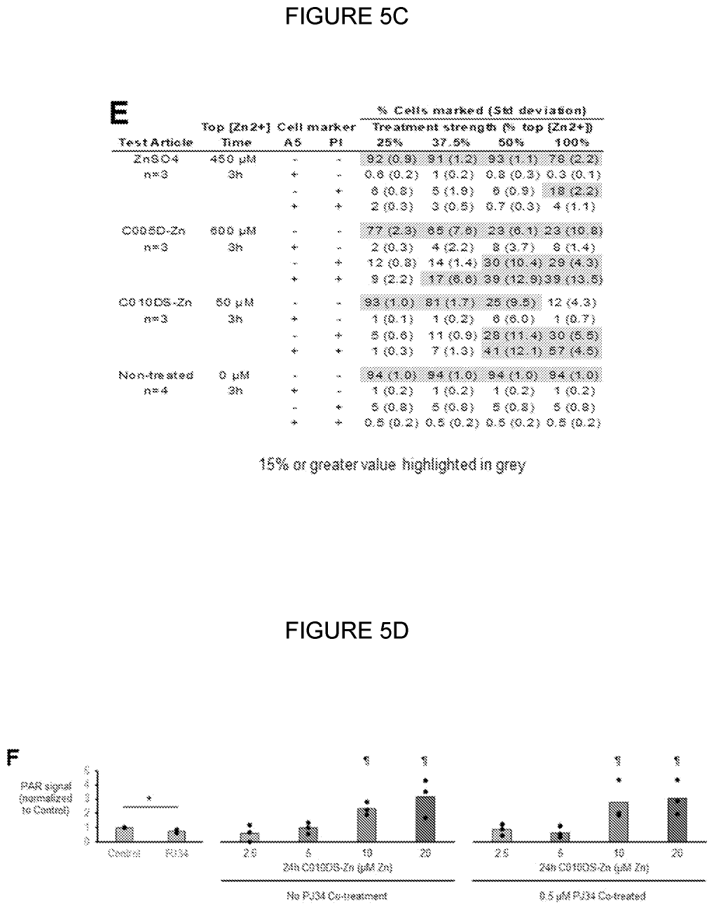

24 h in vitro cytotoxicity was tested for each compound against 4T1 murine triple negative breast cancer cell line in obtaining their IC50₂₄ₕ values using lactate dehydrogenase release assay (LDH assay), which were expressed in zinc concentration (μM Zn) units for comparative viewing. C010DS-Zn showed markedly higher potency at IC50₂₄ₕ of 16.35 μM Zn versus zinc sulfate (80.7 μM Zn) or C005D-Zn (268.7 μM Zn). 7Zn-1Pyr did not show cytotoxic response in the concentration range tested (12.1 μM Zn-300 μM Zn) (FIG. 5A).

Flow cytometry analyses using annexinV (A5) and propidium iodide (PI) markers were performed to compare the in vitro apoptotic/necrotic cell death feature of the observed cytotoxic effects around IC50₂₄ₕ-defined concentration gradient after 3h treatment (FIG. 5C), or over time at an excess concentration over IC50₂₄ₕ (FIG. 5B). 7Zn-1Pyr was excluded from the test as its IC50₂₄ₕ value could not be derived due to its lack of cytotoxicity. The time resolved study treatment showed that C010DS-Zn (50 μM Zn) and zinc sulfate (450 μM Zn) groups induced mainly PI+A5+ and minor PI+ only necrotic cell death 1.5 h after the treatment, while C005D-Zn (600 μM Zn) showed similar necrotic death 3 h after the treatment. A5+ only apoptotic death response was not observed in any of the test groups or at any time points. The concentration-resolved study after 3 h treatment showed that C010DS-Zn, C005D-Zn, and zinc sulfate respectively induced up to 77%, 62%, 26% PI+ only or PI+A5+ necrotic cell death in concentration-dependent manners without notable increases in A5+ only apoptotic cell deaths. Also, the proportion of PI+A5+ versus PI+ only necrosis was found highest in the C010DS-Zn (57% vs 30%) group and the lowest in the zinc sulfate group (4% vs 18%). Collectively, these cytotoxicity tests demonstrated C010DS-Zn was the most potent and efficient inducer of the PI+A5+ necrotic cell death per given zinc content against the 4T1 cells.

### Example 11: In Vitro Mode-of-Action Test

In addition to the cytotoxicity featuring the simultaneous uptake of PI and A5, Fatokun et al. previously defined PARP-dependent nuclear translocation of AIF and subsequent generation of massive DNA breaks as the hallmark features of parthanatos (Parthanatos: mitochondrial-linked mechanisms and therapeutic opportunities. British journal of pharmacology 171, 2000-2016 (2014)). To investigate whether the observed PI+A5 + necrotic 4T1 cell deaths by the zinc compounds (FIG. 5B and 5C) accompanied these features, we performed confocal microscopy studies after 1.5 h treatment for assessing the occurrence of PARP-dependent cytotoxicity, nuclear AIF translocation, and DNA breaks using fluorescent probes of anti-AIF, TUNEL, and Hoechst 33342 (Hoechst) DNA stain upon co-incubation with a potent PARP1 and PARP2 inhibitor PJ34. Assessment of PAR-polymer content, however, could not be incorporated into this experiment due to unavailability of sufficiently sensitive fluorescent probe for PAR-polymer during this study. Also, any longer treatment time could not be applied to this experiment due to extensive cytotoxic changes observed in the C010DS-Zn treated groups.

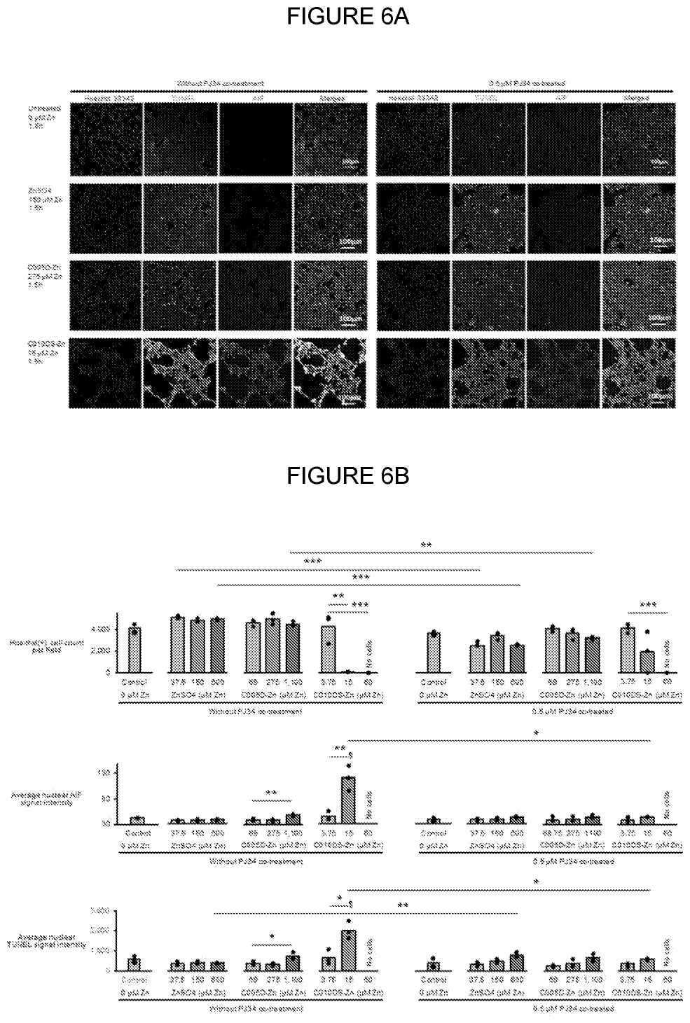

After the 1.5 h treatment, zinc sulfate and C005D-Zn treatment did not cause marked cell detachment across all concentrations tested, although the cell morphologies in the higher concentration treatments showed cytotoxic changes. C010DS-Zn treatment, however, led to complete cell loss in the 60 μM Zn treated group and extensive cell detachments even in the 15 μM Zn treated group. Co-treatment with 0.5 μM PJ34 (a selective PARP1 and PARP2 inhibitor) significantly attenuated the cell detachments in the C010DS-Zn 15 μM Zn group but showed no protective effects in the 60 μM Zn treated group (FIG. 6B).

Subsequent quantitative imaging analysis on the averaged nuclear AIF signal and TUNEL signals, respectively representing the extent of nuclear AIF translocation and the resulting DNA damages in the detected nuclei, showed that C010DS-Zn caused significant nuclear AIF translocation and massive DNA damage induction at 15 μM Zn, which was abrogated by the PJ34 co-treatment. Similar but significantly less trend was seen in the C005D-Zn treated groups. Zinc sulfate treatment, on the other hand, failed to elicit significantly higher nuclear AIF translocation nor the DNA damages.

In further validation of the PARP enzyme involvement observed in the cytotoxicity of C010DS-Zn, we performed PAR-polymer quantification using a commercial ELISA kit and observed that 24 h C010DS-Zn treatment indeed produced dose-dependent accumulation of PAR-polymer indicative of PARP enzyme hyperactivity in the in vitro treated 4T1 cells (FIG. 5D).

As these observations are consistent with the cell death characteristics of parthanatos as stipulated by Fatokun et al., C010DS-Zn was collectively demonstrated as a remarkably stronger inducer of parthanatos over zinc sulfate, C005D-Zn, or other control substances tested.

### Example 12: Ex Vivo Screening Against Patient-Derived Solid Tumors

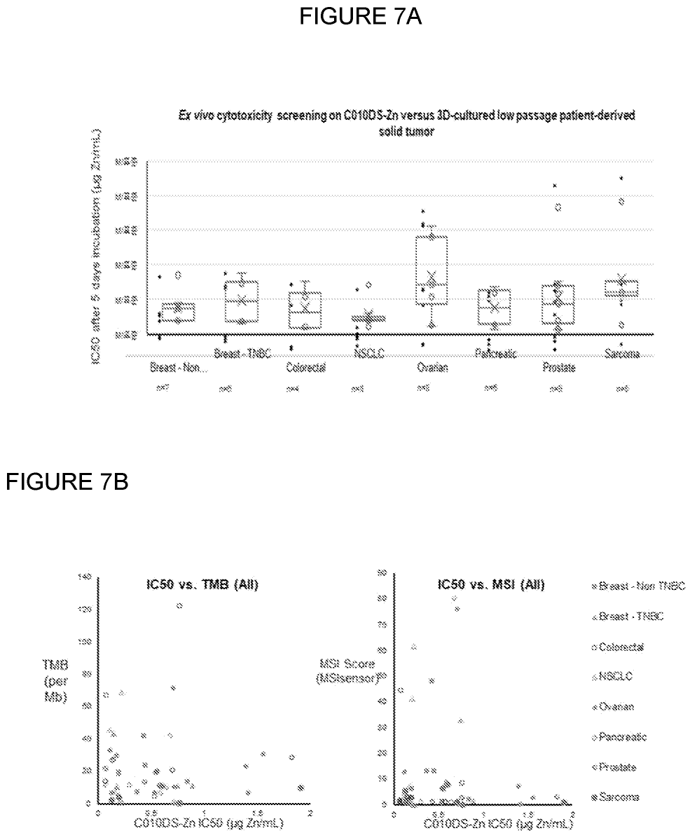

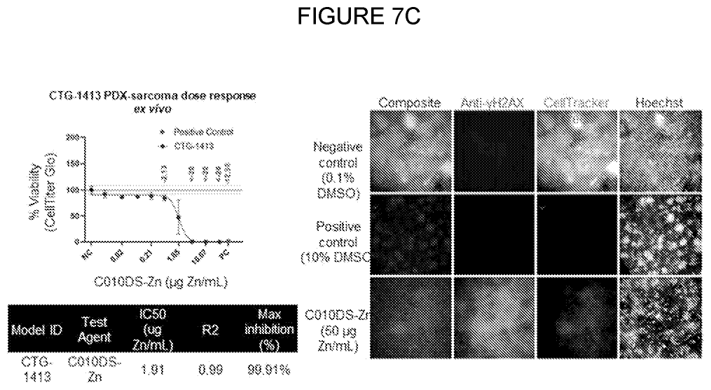

To determine the applicability and consistency of C010DS-Zn pharmacologic activity against wider solid cancer types, we employed the imaging-based ex vivo cytotoxicity screening service platform using the tumor fragments of patient-derived xenografts (PDX) from Champions Oncology/Phenovista. 53 PDX models from 8 cancer types including triple negative breast cancer (BrC-TNBC), non-triple negative breast cancer (BrC-nonTNBC), colorectal cancer (CRC), non-small cell lung cancer (NSCLC), ovarian cancer (OVC), pancreatic cancer (Panc), prostate cancer (PrC), and sarcoma were chosen. C010DS-Zn demonstrated cytotoxicity against all 53 models tested with IC50 values ranging from 0.07 μg Zn/mL to 1.91 μg Zn/mL, with ovarian cancers showing statistically non-significant but higher average IC50 values than other cancer types (FIG. 7A). Statistically significant correlation between C010DS-Zn IC50 values and each model's tumor mutation burden (TMB) or microsatellite instability (MSI) score was also not found, indicating that C010DS-Zn cytotoxicity did not depend on the innate mutational burden of the target models (FIG. 7B) despite the conventional view of DNA-damage mediated PARP activation as the first key step in parthanatos induction (see supra, Fatukun et al.).

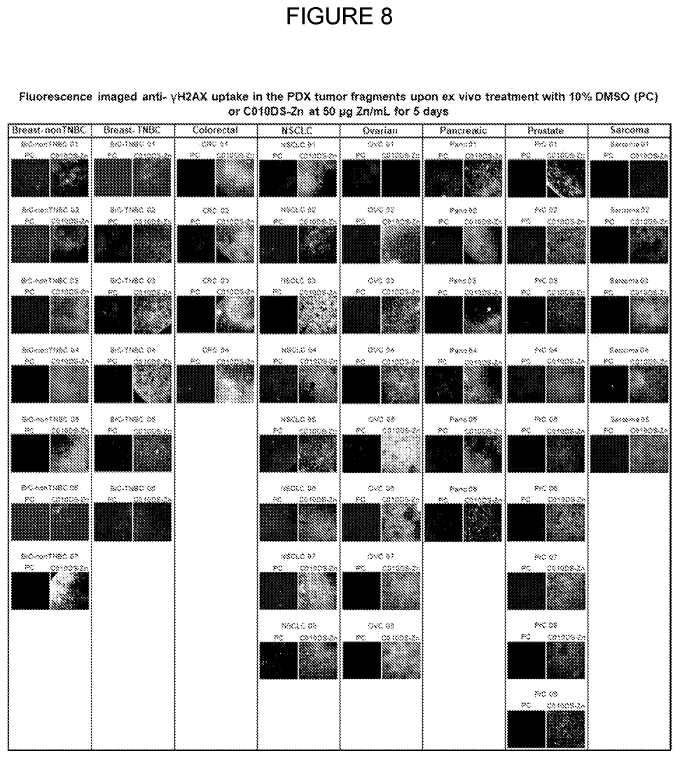

Comparative assessment of the γH2AX level, an indicator of double stranded DNA (dsDNA) breaks, in the tumor fragments between the apoptosis-inducing 10% DMSO positive control treatment and the top-dose C010DS-Zn treatment group showed that C010DS-Zn induced markedly greater dsDNA breaks than the apoptosis positive control, even in the model with the highest IC50 value tested (FIG. 7C). This characteristically massive induction of dsDNA breaks by C010DS-Zn treatment versus the 10% DMSO positive control treatment was further observed in 50 of the 53 ex vivo PDX models tested, indicating high consistency in its pharmacologic action in a diverse set of solid cancer types (FIG. 8).

### Example 13: In Vivo Therapeutic Response to C010DS-Zn Treatment

Next we tested the in vivo therapeutic effects of intravenously (i.v.) administered C010DS-Zn against the immunogenic syngeneic cancer model CT26-Balb/c and immunosuppressive syngeneic cancer model 4T1-Balb/c. Briefly, CT26 is a widely used immunogenic murine colorectal cancer model and a responder to anti-PD1 treatment. 4T1, on the other hand, is a widely used immunosuppressive triple negative breast cancer model that does not respond to anti-PD1 treatment (Kim, K., et al., Eradication of metastatic mouse cancers resistant to immune checkpoint blockade by suppression of myeloid-derived cells. Proc. Nat'l. Acad. Sci. U.S.A. 111, 11774-11779 (2014)).

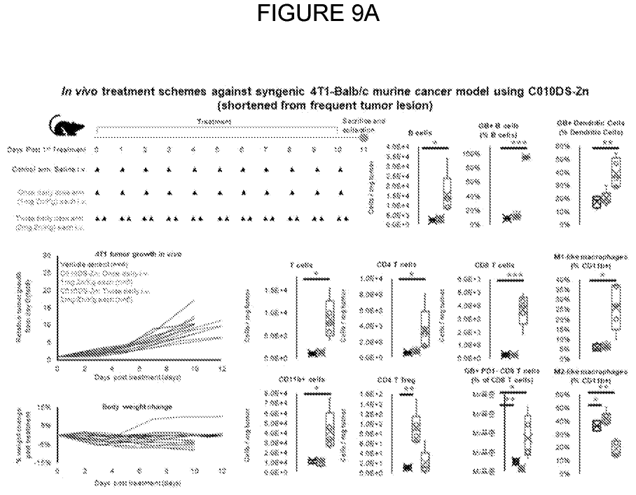

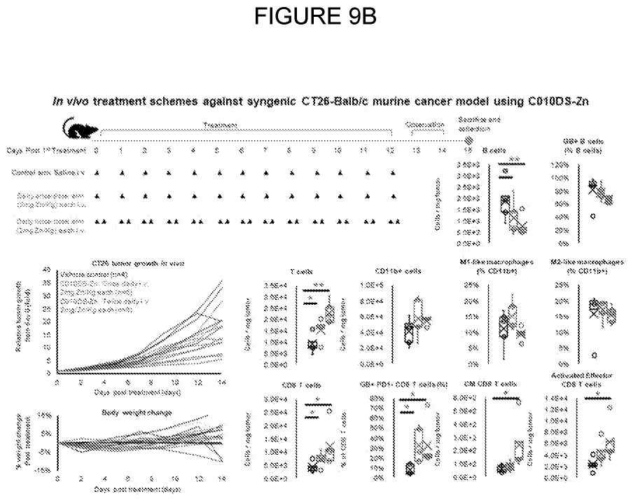

After initial trial-and-error attempts targeting the 4T1-Balb/c model, we discovered that twice daily i.v. administration of C010DS-Zn at each injection dose of 2 mg Zn/Kg was needed in producing observable therapeutic efficacy. 50%+ tumor surface ulcerations problems were frequently encountered during pilot studies in non-treated animals, but toxicity signs meeting the General Toxicity definitions (see below) were not observed during the final study (FIG. 9A). While direct comparison of the tumor growth rate was difficult due to rapid tumor ulceration developments in the vehicle control group (30 mM HEPES at pH 7), end point tumor microenvironment (TME) immune cell analysis revealed that the twice daily injection of 2 mg Zn/Kg C010DS-Zn led to significant immune compartment expansions in CD4 T cells, CD 8 T cells, Granzyme B+ dendritic cells (GB+ DC), and macrophages. Particularly in the macrophage compartment, the proportion of anti-tumor M1-like macrophages was significantly escalated (25% vs Control group's 7%, p<0.05) while that of the pro-tumor M2-like macrophages was suppressed (18% vs Control group's 35%, p<0.005) (FIG. 9A). C010DS-Zn dosing at lesser amount or frequency, including the once daily i.v. at 1 mg Zn/Kg injection, did not produce such results. C010DS-Zn treatments were well tolerated, and the animal body weights remained stable.

C010DS-Zn i.v. treatment against CT26-Balb/c model produced better pronounced and dose-dependent responses in both tumor growth and in the end point TME immune cell analysis. No toxicity signs meeting the General Toxicity definitions were observed during the study (FIG. 9B). While CT26-Balb/c model showed tumor growth reduction in both single and twice daily injection regimens, unlike the 4T1-Balb/c model, significant immune response initiation effect was limited to CD 8 T cell compartment (FIG. 9B). C010DS-Zn treatments were well tolerated, and the animal body weights remained stable.

Interestingly, when comparing the end point tumor immune profile between those of 4T1-Balb/c and CT26-Balb/c control models, we observed that the T cell (1E+3 in 4T1 vs. 9E+3 in CT26), CD11b+ myeloid cells (1E+4 in 4T1 vs. 4E+4 in CT26), and M1 macrophages levels (5% of CD11b+ cells in 4T1 vs. 11% of CD11b+ cells in CT26) were markedly suppressed in the 4T1 model versus the CT26 model. The same comparison between the twice daily C010DS-Zn treated groups of 4T1 and CT26, on the other hand, showed a narrower gap between the two tumor model immune compositions in the T cells (8E+3 in 4T1 vs. 2E+4 in CT26) and CD11b+ cells (4E+4 in 4T1 vs. 5E+4 in CT26). Post treatment M2-like macrophage levels in the 4T1 and the CT26 models were similar at 18% and 15%. Collectively, these observations indicated that the anti-tumor immune initiation effects of the twice daily C010DS-Zn treatments were more pronounced in the tumor-suppressed immune compartments of 4T1-Balb/c and CT26-Balb/c models.

General toxicity. Beginning on Day 0, animals were observed daily and weighed thrice weekly using a digital scale; data including individual and mean gram weights, mean percent weight change versus Day 0 (%vD0) were recorded for each group and %vD0 plotted at study completion. Animals exhibiting 10% weight loss when compared to Day 0, if any, were provided with DietGel™ (ClearH2O®, Westbrook, ME) ad libitum. Animal deaths, if any, were recorded. Groups reporting a mean loss of %vD0 >20 and/or >10% mortality were considered above the maximum tolerated dose (MTD) for that treatment on the evaluated regimen. Additional study toxicity endpoints were mice found moribund or displayed >20% net weight loss for a period lasting 7 days or if the mice displayed >30% net weight loss. Maximum mean %vD0 (weight nadir) for each treatment group was reported at study completion.

### Example 14: Macrophage Clearing Effects of C010DS-Zn at Low Doses

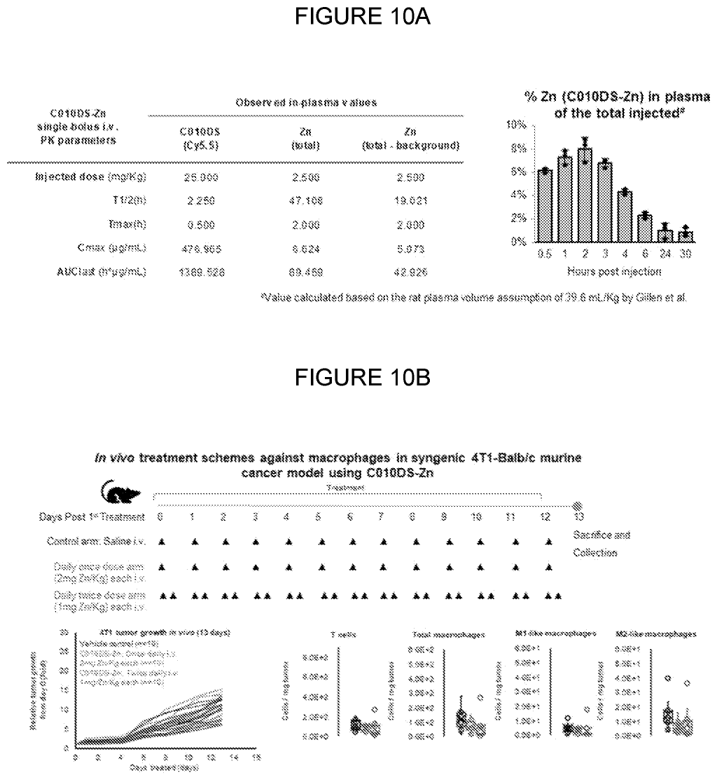

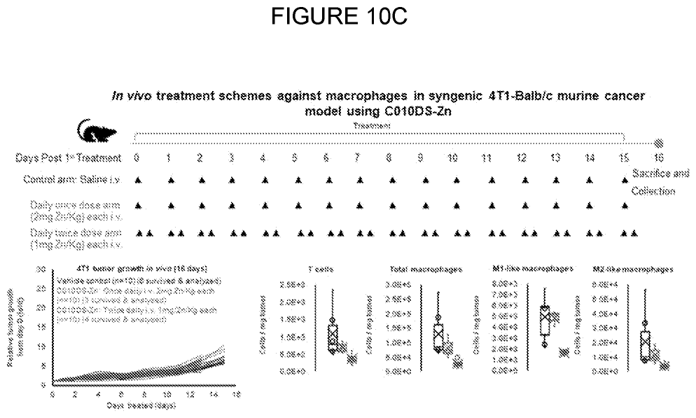

Our observation that twice daily i.v. treatment using C010DS-Zn was needed in producing the anti-tumor immune response initiation in 4T1-Balb/c model prompted us to test the pharmacokinetic profile of i.v. injected C010DS-Zn (FIG. 10A). Evaluation of in-rat PK profile after a single bolus injected i.v. C010DS-Zn in non-tumor bearing Sprague-Dawley Rats revealed that only a fraction of the zinc injected by C010DS-Zn was found in the cell-free plasma compartment of blood with Tₘₐₓ of 2 h, indicating rapid systemic clearance of the injected C010DS-Zn by mononuclear phagocytic system (MPS) (Song, G., et al., Nanoparticles and the mononuclear phagocyte system: pharmacokinetics and applications for inflammatory diseases. Curr. Rheumatol. Rev. 10, 22-34 (2014)). As MPS mainly consists of hepatic and splenic macrophages, this observation suggested possible direct cytotoxic interaction of C010DS-Zn against macrophages.

To assess whether C010DS-Zn was capable of directly reducing live macrophages in the TME, we decided to test its effects on the live TME macrophage populations at lower doses that would not produce anti-tumor activity or immune initiation effects. And as 4T1 cancer model was reported to undergo TME immunity transition from early immune-suppressive state to metastasis-promoting M2-like macrophage enriched state in the late phase, we decided to characterize the TME immunity at 13 days post treatment and 16 days post treatment. (Steenbrugge, J., et al., Anti-inflammatory signaling by mammary tumor cells mediates prometastatic macrophage polarization in an innovative intraductal mouse model for triple-negative breast cancer. J. Exp. Clin. Cancer Res. 37, 191 (2018); Yang, Y., et al., Celastrol inhibits cancer metastasis by suppressing M2-like polarization of macrophages. Biochem. Biophys. Res. Comm. 503, 414-419 (2018).) Consistent with the previous reports, 4T1 tumors collected on day 13 was characterized with greatly suppressed T cells and macrophage contents of both M1-like and M2-like types. In these animals, no toxicity signs meeting the General Toxicity definitions (see Example 13) were observed during the study (FIG. 10B). Those harvested on day 16, on the contrary, contained greatly increased amounts of macrophages (1E+2 in 13 days tumor vs. 1.3E+5 in 16 days tumor), mainly due to the expansion in the M2-like macrophage population, together with greatly expanded T cell content suggestive of active inflammation (FIG. 10B and FIG. 10C). Many of the 16-day sacrificed animals developed 50%+ tumor surface ulceration toward the end of the study, and hence were declared moribund before day 16. When treated with the lowered C010DS-Zn doses at once daily i.v. injection of 2 mg Zn/Kg or twice daily injections of 1 mg Zn/Kg each, the treatments led to significantly reduced macrophage content in the TME in both 13 days and 16 days treated mice. Notably in the 16 days treated groups, once daily dose of 2 mg Zn/Kg led to preferential reduction in the M2-like macrophages, while twice daily doses of 1 mg Zn/Kg each led to marked reduction in both M1-like and M2-like macrophages. Both C010DS-Zn treated groups of the 16 days mice also showed markedly reduced level of T cell content in the TME, suggesting reduced inflammation (FIG. 10C).

In the foregoing disclosure, the singular forms “a,” “an,” and “the” include plural referents unless stated, or the context requires, otherwise. Thus, these articles may mean “one,” “one or more,” or “one or more than one,” unless the full scope of these terms would be excluded by the context. Where alternatives are introduced by the term “or,” unless the alternatives are mutually exclusive options the term “or” should be read to include the meaning “and/or.” Even though the term “and/or” may be used in this disclosure, that is done for emphasis and not imply that “or” should solely be read as disjunctive

Although the invention is disclosed herein with respect to particular embodiments and examples, the invention can be embodied in other specific forms without departing from the spirit or essential characteristics thereof. The description and figures thus should be regarded as illustrative and not to be construed to limit the invention. The scope of the invention is determined by the claims, and all appropriate modifications and equivalents that lie within the claims are embraced therein.

I claim:

1. A polymer conjugate comprising:
a polymer backbone comprising polyglutamic acid, which comprises glutamic acid monomer units joined by peptide bonds;
a plurality of first receptor-targeting moieties, each covalently bonded to and pendant from a monomer unit in the polymer backbone via a first linker moiety;
a plurality of ionophores, each covalently bonded to and pendant from a monomer unit in the polymer backbone via a cleavable linker moiety;
or a salt or solvate thereof.

2. The polymer conjugate of claim 1, further comprising:
a plurality of second receptor-targeting moieties, each covalently bonded to and pendant from a monomer unit in the polymer backbone via a second linker moiety.

3. The polymer conjugate of claim 1 or 2, further comprising:
a plurality of labeling moieties, each covalently bonded to and pendant from a monomer unit in the polymer backbone via a label-linker moiety.

4. The polymer conjugate of claim 1, 2, or 3, wherein the salt is a zinc(II) complex thereof.

5. The polymer conjugate of claim 4, wherein the weight-to-weight ratio of zinc(II) to the polymer conjugate is in the range of 1:5 to 1:20.

6. The polymer conjugate of claim 4, wherein the number of zinc(II) ions per polymer conjugate on average is at least 20.

7. A polymer conjugate according to Formula IV, V, or VI: 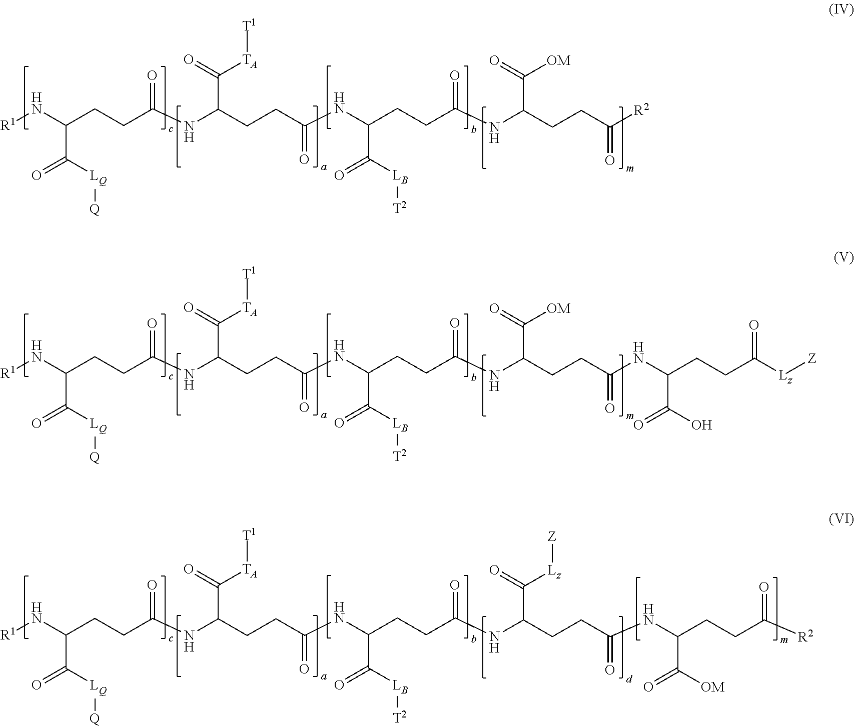
or a metal ion complex thereof, wherein:
R¹ is H;
R² is OH or OM or L_{A}-T¹ or L_{B}-T² or L_{Q}-Q;
L_{A} is a bond or bifunctional linking group having a first terminal site bonded to the polymer and a second terminal site bonded to T¹ , wherein the first terminal site and the second terminal site are connected through a chain of 3 to 20 atoms;
L_{B} is a bond or bifunctional linking group having a first terminal site bonded to the polymer and a second terminal site bonded to T²,
wherein the first terminal site and the second terminal site are connected through a chain of 3 to 20 atoms;
L_{Q} is bifunctional linking group having a first terminal site bonded to the polymer and a cleavable terminal site bonded to Q, wherein the first terminal site and the cleavable terminal site are connected through a chain of 3 to 20 atoms;
L_{Z}, when present, is a bond or bifunctional linking group having a first terminal site bonded to the polymer and a second terminal site bonded to Z, wherein the first terminal site and the second terminal site are connected through a chain of 3 to 20 atoms;
T¹ is a first targeting moiety;
T² is a second targeting moiety;
Q is an ionophore moiety;
Z, when present, is fluorophore moiety;
each instance of M is independently H, a proton, an alkali ion; a pharmaceutically acceptable monovalent cation, or absent;
a and b are zero or a finite number up to about 5, but a and b are not both zero;
c is a finite number in the range from about 3 to about 50;
d is, when present, approximately 1; and
m is a finite number in the range from about 50 to about 700.

8. A pharmaceutical composition comprising a polymer conjugate according to claim 7 and a pharmaceutically-acceptable diluent, carrier, buffer, vehicle, or any combination thereof.

9. The pharmaceutical composition of claim 8, wherein the pharmaceutical composition is formulated for parenteral administration.

10. The pharmaceutical composition of claim 8, wherein the polymer conjugate is a zinc(II) complex of the polymer conjugate.

11. The pharmaceutical composition of claim 9, wherein the weight-to-weight ratio of zinc(II) to the polymer conjugate is in the range of 1:5 to 1:20.

12. The pharmaceutical composition of claim 9, wherein the number of zinc(II) ions per polymer conjugate on average is at least 20.

13. A method for treating a tumor in a patient comprising administering to said patient a therapeutically effective amount of the pharmaceutical composition according to any one of claims 8 to 12.

14. A method of inducing parthanatos in a tumor in a patient, the method comprising administering to said patient a therapeutically effective amount of the pharmaceutical composition according to any one of claims 8 to 12.
